(19) United States
(12) Patent Application Publication
(10) Pub. No.: US 2025/0051359 A1
(43) Pub. Date: Feb. 13, 2025

(54) MACROCYCLIC 2-AMINO-BUT-3-ENAMIDES AS INHIBITORS OF MCL-1

(71) Applicant: JANSSEN PHARMACEUTICA NV, Beerse (BE)

(72) Inventors: Soufyan JERHAOUI, Turnhout (BE); Gaston Stanislas M. DIELS, Turnhout (BE)

(21) Appl. No.: 18/710,719

(22) PCT Filed: Nov. 15, 2022
(86) PCT No.: PCT/EP2022/081981

§ 371 Date: May 16, 2024

(30) Foreign Application Priority Data
Nov. 16, 2021 (EP) 21208372.9

(51) Int. Cl. C07D 513/14; A61K 31/553

(52) U.S. Cl. CPC C07D 513/14; A61K 31/553

(57) ABSTRACT

The present invention relates to pharmaceutical agents useful for therapy and/or prophylaxis in a subject, pharmaceutical composition comprising such compounds, and their use as MCL-1 inhibitors, useful for treating diseases such as cancer.

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cylosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009; (113) 2805-2815).
WO2019046150 discloses macrocyclic compounds that inhibit mcl-1 protein.
WO2016033486 discloses tetrahydronaphthalene derivatives that inhibit mcl-1 protein.
WO2019036575, WO2017147410, and WO2018183418 disclose compounds that inhibit mcl-1 protein.
WO2019222112 discloses MCL-1 inhibitors for treating cancer.
WO2020097577 discloses spiro-sulfonamide derivatives as inhibitors of myeloid cell leukemia-1 (MCL-1) protein.
WO2021021259 describes formulations and dosages for administering a compound that inhibits MCL1 protein.
WO2019173181 discloses MCL-1 inhibitors.
WO2021211922 discloses spiro-sulfonimidamide derivatives as inhibitors of myeloid cell leukemia-1 (mcl-1) protein.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AMIL), and acute lymphoblastic leukemia (ALL).

### SUMMARY OF THE INVENTION

The present invention concerns compounds of Formula (I):

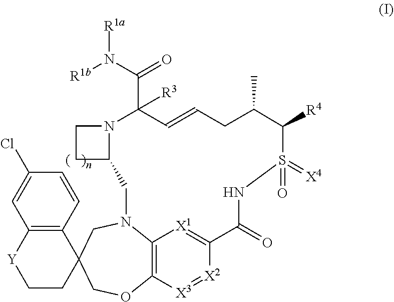

wherein
R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹, wherein said C₁₋₆alkyl, C₂₋₆alkenyl, C₂₋₆alkynyl, C₃₋₇cycloalkyl or C₃₋₇cycloalkenyl is optionally substituted with one or two R²;
or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of OR^{f}, SR^{f}, CN and halo;
or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of OR^{f}, SR^{f}, CN and halo;
each R² is independently selected from the group consisting of OR^{f}, SR^{f}, CN, halo, CF₃, NR^{m}Rⁿ, SO₂R^{c}, C(═O)R^{c}, C(═O)OR^{d}, C(═O)NR^{d}R^{e}, SO₂NR^{d}R^{e}, C₃₋₇cycloalkyl, C₃₋₇cycloalkenyl, Het¹, Ar¹, Het², and Cy¹, wherein said C₃₋₇cycloalkyl or C₃₋₇cycloalkenyl is optionally substituted with one or two substituents each independently selected from the group consisting of OR^{f}, SR^{f}, CN, halo and NR^{d}R^{e};
R^{c} is selected from the group consisting of C₁₋₆alkyl, C₃₋₇cycloalkyl, Het¹, Ar¹ and Het²;
R^{m} and Rⁿ are each independently selected from the group consisting of hydrogen, methyl, C₂₋₇alkyl, C₃₋₇cycloalkyl, Het¹, Ar¹, and Het², wherein said C₂₋₇alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;
R^{d} and R are each independently selected from the group consisting of hydrogen, methyl, C₂₋₇alkyl, C₃₋₇cycloalkyl, Het¹, Ar¹, and Het², wherein said C₂₋₇alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;
or R^{d} and R^{e} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
or R^{d} and R^{e} are taken together to form together with the N-atom to which they are attached a fused 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
n is 1 or 2;
R^{f} is selected from the group consisting of hydrogen, C₁₋₆alkyl, CF₃, C₃₋₇cycloalkyl, Het¹, Ar¹, Het², wherein said C₁₋₆alkyl or C₃₋₇cycloalkyl is optionally substituted with one substituent selected from the group consisting of ORⁱ, SRⁱ, CN, halo, NR^{m}Rⁿ, SO₂R^{c}, C(═O)R^{c}, C(═O)OR^{d}, C(═O)NR^{d}R^{e}, SO₂NR^{d}R^{e}, C₃₋₇cycloalkyl, Het¹, Ar¹ and Het²;
R^{g} and R^{h} are each independently selected from the group consisting of hydrogen, C₁₋₆alkyl and C₃₋₇cycloalkyl;
or R^{g} and R^{h} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;
Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{k}, CN, halo, CF₃, and C₁₋₄alkyl optionally substituted with one substituent selected from the group consisting of ORⁱ, SRⁱ, CN and halo;
Het² represents a 5- to 6-membered monocyclic aromatic ring containing one, two, three or four heteroatoms each independently selected from O, S, and N, wherein said S-atom might be substituted to form S(═O) or S(═O)₂, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Ar¹ represents phenyl optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
Rⁱ represents hydrogen, C₁₋₆alkyl or C₃₋₇cycloalkyl;
R^{j} and R^{k} are each independently selected from the group consisting of hydrogen, C₁₋₆alkyl and C₃₋₇cycloalkyl;
R³ represents hydrogen, C₁₋₄alkyl or C₁₋₄alkyl-OH;
R⁴ represents hydrogen or methyl;
R⁵ represents —(C═O)-phenyl, —(C═O)-Het⁴ or —(C═O)-Het³; wherein said phenyl, Het³ or
Het⁴ are optionally substituted with one or two substituents selected from methyl or methoxy;
Het⁴ represents a C-linked 4- to 7-membered monocyclic fully saturated heterocyclyl containing one or two heteroatoms each independently selected from O, S, and N; wherein said S-atom might be substituted to form S(═O) or S(═O)₂;
Het³ represents a C-linked 5- or 6-membered monocyclic aromatic ring containing one, two or three heteroatoms each independently selected from O, S, and N;
Y represents O or CH₂;
X¹ represents CR⁶;
X² represents CR⁷;
X³ represents CR⁸;
R⁶, R⁷ and R⁸ each independently represent hydrogen, fluoro or chloro;
X⁴ represents O or NR⁵;
and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₇alkyl’ as used herein as a group or part of a group represents a straight or branched chain fully saturated hydrocarbon radical having from 2 to 7 carbon atoms, such as ethyl, n-propyl, isopropyl, n-butyl, s-butyl, t-butyl, n-pentyl, n-hexyl and the like.

The term ‘C₃₋₇cycloalkyl’ as used herein as a group or part of a group defines a fully saturated, cyclic hydrocarbon radical having from 3 to 7 carbon atoms, such as cyclopropyl, cyclobutyl, cyclopentyl and cyclohexyl.

The term “C₂₋₆alkenyl” as used herein as a group or part of a group represents a double bond containing straight or branched chain hydrocarbon radical having from 2 to 6 carbon atoms, such as ethenyl, propenyl, isopropenyl, buten-1-yl, (2Z)-buten-2-yl, (2E)-buten-2-yl, buten-3-yl, 2-methylpropen-1-yl, 1,3-butadiene, penten-1-yl, (2Z)-penten-2-yl, (2E)-penten-2-yl, (3Z)-penten-3-yl, (3E)-penten-3-yl, (4Z)-penten-4-yl, (4E)-penten-4-yl, penten-5-yl and the like.

The term “C₃₋₇cycloalkenyl” as used herein as a group or part of a group defines a double bond containing cyclic hydrocarbon radical having from 3 to 7 carbon atoms, such as cyclopropenyl, cyclobutenyl, cyclopentenyl, cyclohexenyl and cycloheptenyl.

The term “C₂₋₆alkynyl” as used herein as a group or part of a group represents a triple bond containing straight or branched chain hydrocarbon radical having from 2 to 6 carbon atoms, such as ethynyl, 1-propynyl, 2-propynyl, butyn-1-yl, butyn-2-yl, butyn-3-yl, 1,3-butadiyne, pentyn-1-yl, pentyn-2-yl, pentyn-3-yl, pentyn-5-yl and the like.

It will be clear for the skilled person that S(═O)₂ or SO₂ represents a sulfonyl moiety.

It will be clear for the skilled person that CO or C(═O) represents a carbonyl moiety.

Non-limiting examples of two R groups taken together to form together with the N-atom to which they are attached a 4- to 7-membered monocyclic fully saturated heterocyclyl containing one N-atom and optionally one additional heteroatom selected from O, S, and N, include, but are not limited to N-linked azetidinyl, N-linked pyrrolidinyl, N-linked morpholinyl, N-linked piperazinyl, and N-linked piperidinyl.

Non-limiting examples of 4- to 7-membered monocyclic fully saturated heterocyclyl containing one or two heteroatoms each independently selected from O, S, and N, include, but are not limited to tetrahydropyranyl, piperazinyl, tetrahydrofuranyl, 1,4-dioxanyl, tetrahydropyranyl, 1,4-oxazepanyl, 1,3-dioxolanyl, morpholinyl and azetidinyl.

Non-limiting examples of C-linked 4- to 7-membered monocyclic fully saturated heterocyclyl containing one or two heteroatoms each independently selected from O, S, and N, include, but are not limited to C-linked tetrahydropyranyl, C-linked piperazinyl, C-linked tetrahydrofuranyl, C-linked 1,4-dioxanyl, C-linked tetrahydropyranyl, C-linked 1,4-oxazepanyl, C-linked 1,3-dioxolanyl, C-linked morpholinyl and C-linked azetidinyl.

Within the context of this invention, bicyclic 6- to 11-membered bicyclic fully saturated heterocyclyl groups, or 6- to 11-membered bicyclic fully saturated ring systems, include fused, spiro and bridged bicycles.

Fused bicyclic groups are two cycles that share two atoms and the bond between these atoms.

Spiro bicyclic groups are two cycles that are joined at a single atom.

Bridged bicyclic groups are two cycles that share more than two atoms.

Examples of 6- to 11-membered bicyclic fully saturated ring systems optionally containing one or two heteroatoms each independently selected from O, S, and N, include, but are not limited to

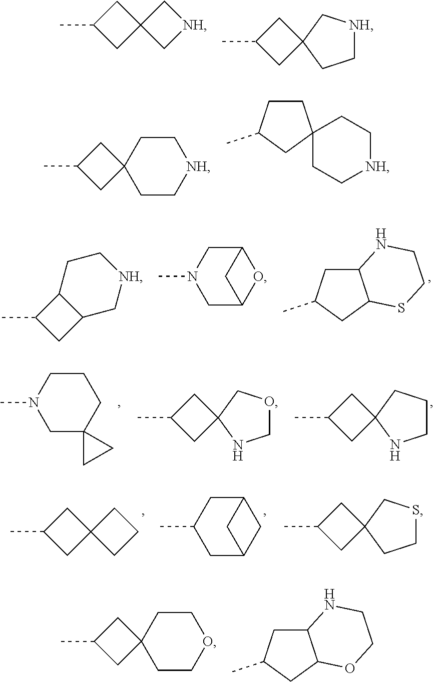

and the like.

Examples of two R groups taken together to form together with the N-atom to which they are attached a 6- to 11-membered bicyclic fully saturated heterocyclyl containing one N-atom and optionally one or two additional heteroatoms each independently selected from O, S, and N, include, but are not limited to

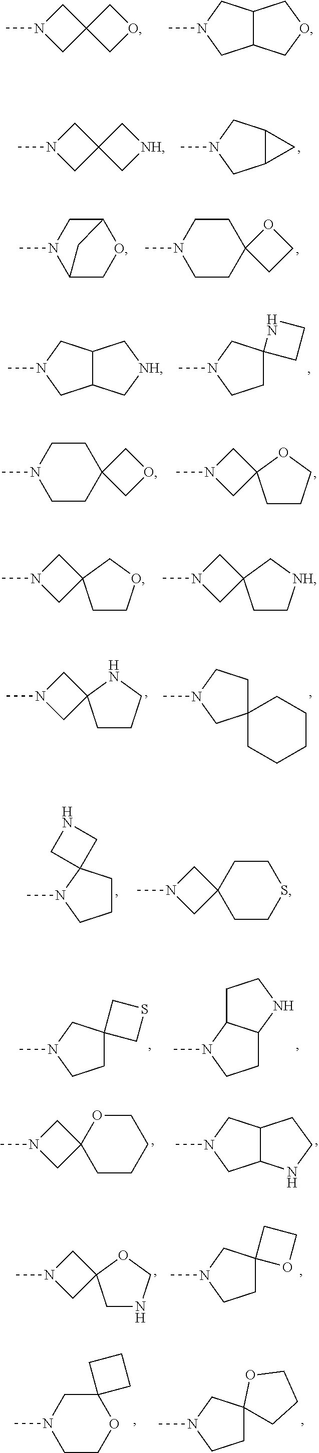

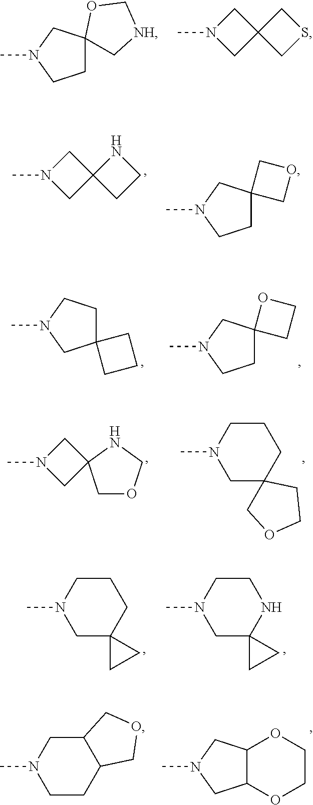

and the like.

Non-limiting examples of 5- to 6-membered monocyclic aromatic ring containing one or two heteroatoms each independently selected from O, S, and N, include, but are not limited to

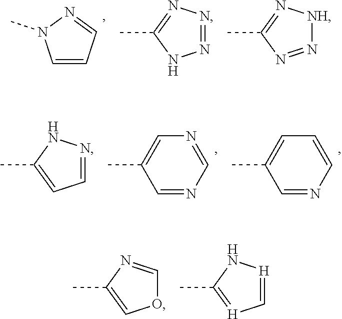

and the like.

Non-limiting examples of C-linked 5- or 6-membered monocyclic aromatic ring containing one or two heteroatoms each independently selected from O, S, and N, include, but are not limited to

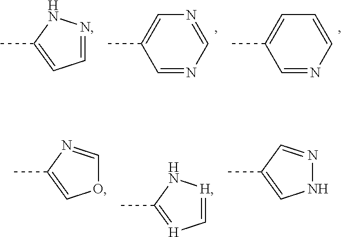

and the like.

Unless otherwise specified or clear from the context, heterocyclyl groups (e.g. Het¹), aromatic rings containing an heteroatom (e.g. Het²), or ring systems containing an heteroatom (e.g. Cy¹), can be attached to the remainder of the molecule of Formula (I) through any available ring carbon atom (C-linked) or nitrogen atom (N-linked) if available.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

When any variable occurs more than one time in any constituent, each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers. Where the stereochemistry of any particular chiral atom is not specified in the structures shown herein, then all stereoisomers are contemplated and included as the compounds of the invention, either as a pure stereoisomer or as a mixture of two or more stereoisomers.

Hereinbefore and hereinafter, the term “compound of Formula (I)” is meant to include the stereoisomers thereof and the tautomeric forms thereof. However where stereochemistry, as mentioned in the previous paragraph, is specified by bonds which are shown as solid wedged or hashed wedged bonds, or are otherwise indicated as having a particular configuration (e.g. R, S), or when the stereochemistry around a double bond is shown (e.g. in Formula (I)), then that stereoisomer is so specified and defined. It will be clear this also applies to subgroups of Formula (I).

It follows that a single compound may, where possible, exist in both stereoisomeric and tautomeric form.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other.

A 1:1 mixture of a pair of enantiomers is a racemate or racemic mixture.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, diastereomers, racemates, E isomers, Z isomers, cis isomers, trans isomers and mixtures thereof, unless the context indicates otherwise and whenever chemically possible.

The meaning of all those terms, i.e. enantiomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, N-oxides and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C ¹³N, ¹⁵O, ¹⁷O, ¹⁸O ³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

In an embodiment, the present invention concerns novel compounds of Formula (I), wherein
R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹, wherein said C₁₋₆alkyl, C₂₋₆alkenyl, C₂₋₆alkynyl, C₃₋₇cycloalkyl or C₃₋₇cycloalkenyl is optionally substituted with one or two R²;
or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of OR^{f}, SR^{f}, CN and halo;
or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of OR^{f}, SR^{f}, CN and halo;
each R² is independently selected from the group consisting of OR^{f}, SR^{f}, CN, halo, CF₃, NR^{m}Rⁿ, SO₂R^{c}, C(═O)R^{c}, C(═O)OR^{d}, C(═O)NR^{d}R^{e}, SO₂NR^{d}R^{e}, C₃₋₇cycloalkyl, C₃₋₇cycloalkenyl, Het¹, Ar¹, Het², and Cy¹, wherein said C₃₋₇cycloalkyl or C₃₋₇cycloalkenyl is optionally substituted with one or two substituents each independently selected from the group consisting of OR^{f}, SR^{f}, CN, halo and NR^{d}R^{e};
R^{c} is selected from the group consisting of C₁₋₆alkyl, C₃₋₇cycloalkyl, Het¹, Ar¹ and Het²;
R^{m} and Rⁿ are each independently selected from the group consisting of hydrogen, methyl, C₂₋₇alkyl, C₃₋₇cycloalkyl, Het¹, Ar¹, and Het², wherein said C₂₋₇alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;
R^{d} and Rare each independently selected from the group consisting of hydrogen, methyl, C₂₋₇alkyl, C₃₋₇cycloalkyl, Het¹, Ar¹, and Het², wherein said C₂₋₇alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;
or R^{d} and R^{e} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo; or R^{d} and R^{e} are taken together to form together with the N-atom to which they are attached a fused 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
n is 1 or 2;
R^{f} is selected from the group consisting of hydrogen, C₁₋₆alkyl, CF₃, C₃₋₇cycloalkyl, Het¹, Ar¹, Het², wherein said C₁₋₆alkyl or C₃₋₇cycloalkyl is optionally substituted with one substituent selected from the group consisting of ORⁱ, SRⁱ, CN, halo, NR^{m}Rⁿ, SO₂R^{c}, C(═O)R^{c}, C(═O)OR^{d}, C(═O)NR^{d}R^{e}, SO₂NR^{d}R^{e}, C₃₋₇cycloalkyl, Het¹, Ar¹ and Het²;
R^{g} and R^{h} are each independently selected from the group consisting of hydrogen, C₁₋₆alkyl and C₃₋₇cycloalkyl;
or R^{g} and R^{h} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;
Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Het² represents a 5- to 6-membered monocyclic aromatic ring containing one, two, three or four heteroatoms each independently selected from O, S, and N, wherein said S-atom might be substituted to form S(═O) or S(═O)₂, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Ar¹ represents phenyl optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
Rⁱ represents hydrogen, C₁₋₆alkyl or C₃₋₇cycloalkyl;
R^{j} and R^{k} are each independently selected from the group consisting of hydrogen, C₁₋₆alkyl and C₃₋₇cycloalkyl;
R³ represents hydrogen, C₁₋₄alkyl or C₁₋₄alkyl-OH;
R⁴ represents methyl;
Y represents O or CH₂;
X¹ represents CR⁶;
X² represents CR⁷;
X³ represents CR⁸;
R⁶, R⁷ and R⁸ each independently represent hydrogen, fluoro or chloro;
X⁴ represents O;
and the pharmaceutically acceptable salts and the solvates thereof.

In an embodiment, the present invention concerns novel compounds of Formula (I), wherein
R^{1a} and R^{1b} represent C₁₋₆alkyl optionally substituted with one or two R²;
or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;
each R² is independently selected from the group consisting of OR^{f} and Het¹;
n is 1 or 2;
R^{f} represents C₁₋₆alkyl;
Het¹ represents a 4- to 7-membered monocyclic fully saturated heterocyclyl containing one or two heteroatoms each independently selected from O, S, and N, wherein said S-atom might be substituted to form S(═O) or S(═O)₂;
R³ represents hydrogen;
R⁴ represents methyl;
Y represents CH₂;
X¹ represents CR⁶;
X² represents CR⁷;
X³ represents CR⁸;
R⁶, R⁷ and R⁸ represent hydrogen;
X⁴ represents O;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein R^{1a} represents methyl.

In an embodiment, the present invention relates to those compounds of Formula (I) and the pharmaceutically acceptable salts, and the solvates thereof, or any subgroup thereof as mentioned in any of the other embodiments, wherein Y represents O.

In an embodiment, the present invention relates to those compounds of Formula (I) and the pharmaceutically acceptable salts, and the solvates thereof, or any subgroup thereof as mentioned in any of the other embodiments, wherein Y represents CH₂.

In an embodiment, the present invention relates to those compounds of Formula (I) and the pharmaceutically acceptable salts, and the solvates thereof, or any subgroup thereof as mentioned in any of the other embodiments, wherein n is 1.

In an embodiment, the present invention relates to those compounds of Formula (I) and the pharmaceutically acceptable salts, and the solvates thereof, or any subgroup thereof as mentioned in any of the other embodiments, wherein n is 2.

In an embodiment, the present invention relates to those compounds of Formula (I) and the pharmaceutically acceptable salts, and the solvates thereof, or any subgroup thereof as mentioned in any of the other embodiments, wherein R^{1a} is not taken together with R^{1b} to form a heterocyclyl.

In an embodiment, the present invention relates to those compounds of Formula (I) and the pharmaceutically acceptable salts, and the solvates thereof, or any subgroup thereof as mentioned in any of the other embodiments, wherein R^{d} is not taken together with R^{e} to form a heterocyclyl.

In an embodiment, the present invention relates to those compounds of Formula (I) and the pharmaceutically acceptable salts, and the solvates thereof, or any subgroup thereof as mentioned in any of the other embodiments, wherein R^{1a} is taken together with R^{1b}.

In an embodiment, the present invention relates to those compounds of Formula (I) and the pharmaceutically acceptable salts, and the solvates thereof, or any subgroup thereof as mentioned in any of the other embodiments, wherein R^{1a} and R^{1b} are taken together to form together with the N-atom to which they are attached a heterocyclyl as defined in any one of the other embodiments.

In an embodiment, the present invention relates to those compounds of Formula (I) and the pharmaceutically acceptable salts, and the solvates thereof, or any subgroup thereof as mentioned in any of the other embodiments, wherein R^{1a} and R^{1b} are taken together to form together with the N-atom to which they are attached a monocyclic heterocyclyl as defined in any one of the other embodiments.

In an embodiment, the present invention relates to those compounds of Formula (I) and the pharmaceutically acceptable salts, and the solvates thereof, or any subgroup thereof as mentioned in any of the other embodiments, wherein R^{1a} and R^{1b} are taken together to form together with the N-atom to which they are attached a bicyclic heterocyclyl as defined in any one of the other embodiments.

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹,
wherein said C₁₋₆alkyl, C₂₋₆alkenyl, C₂₋₆alkynyl, C₃₋₇cycloalkyl or C₃₋₇cycloalkenyl is optionally substituted with one or two R².

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of
C₁₋₆alkyl, C₂₋₆alkenyl, C₂₋₆alkynyl, C₃₋₇cycloalkyl, C₃₋₇cycloalkenyl, Het¹, Ar¹, Het², and Cy¹,
wherein said C₁₋₆alkyl, C₂₋₆alkenyl, C₂₋₆alkynyl, C₃₋₇cycloalkyl or C₃₋₇cycloalkenyl is optionally substituted with one or two R².

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of
C₁₋₆alkyl, C₂₋₆alkenyl, C₂₋₆alkynyl, C₃₋₇cycloalkyl, and C₃₋₇cycloalkenyl,
wherein said C₁₋₆alkyl, C₂₋₆alkenyl, C₂₋₆alkynyl, C₃₋₇cycloalkyl or C₃₋₇cycloalkenyl is optionally substituted with one or two R².

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of
C₁₋₆alkyl optionally substituted with one R².

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of
Het¹, Ar¹, and C₁₋₆alkyl optionally substituted with one R².

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of
Het¹, Ar¹, and C₁₋₆alkyl optionally substituted with one R², and
R² is selected from the group consisting of OR^{f}, CF₃, NR^{m}Rⁿ, SO₂R^{c}, Het¹, and Het².

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of
C₁₋₆alkyl optionally substituted with one R²; and
R² is selected from the group consisting of OR^{f} and Het¹.

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are each independently selected from the group consisting of
C₁₋₆alkyl optionally substituted with one R²; and
R² is selected from the group consisting of OR^{f} and Het¹; and
n is 2.

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are not hydrogen.

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of OR^{f}, SR^{f}, CN and halo.

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of OR^{f}, SR^{f}, CN and halo.

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said heterocyclyl is optionally substituted with one or two substituents each independently selected from the group consisting of OR^{f}, NR^{d}R^{e}, CN, halo, CF₃, and C₁₋₄alkyl optionally substituted with one substituent selected from the group consisting of OR^{f} and CN.

In an embodiment, the present invention relates to those compounds of Formula (I) and the pharmaceutically acceptable salts, and the solvates thereof, or any subgroup thereof as mentioned in any of the other embodiments, wherein
R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said heterocyclyl is optionally substituted with one or two substituents each independently selected from the group consisting of halo and C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein each R² is independently selected from the group consisting of OR^{f}, CF₃, NR^{m}Rⁿ, SO₂R^{c}, Het¹, and Het².

In an embodiment, the present invention relates to those compounds of Formula (I) and the pharmaceutically acceptable salts, and the solvates thereof, or any subgroup thereof as mentioned in any of the other embodiments, wherein
R³ represents hydrogen; R⁴ represents methyl; and X⁴ represents O.

In an embodiment, the present invention relates to those compounds of Formula (I) and the pharmaceutically acceptable salts, and the solvates thereof, or any subgroup thereof as mentioned in any of the other embodiments, wherein
R⁴ represents methyl; and X⁴ represents O.

In an embodiment, the present invention relates to those compounds of Formula (I) and the pharmaceutically acceptable salts, and the solvates thereof, or any subgroup thereof as mentioned in any of the other embodiments, wherein X⁴ represents O.

In an embodiment, the present invention relates to those compounds of Formula (I) and the pharmaceutically acceptable salts, and the solvates thereof, or any subgroup thereof as mentioned in any of the other embodiments, wherein X⁴ represents NR⁵.

In an embodiment, the present invention relates to those compounds of Formula (I) and the pharmaceutically acceptable salts, and the solvates thereof, or any subgroup thereof as mentioned in any of the other embodiments, wherein R⁶, R⁷ and R⁸ each independently represent hydrogen or fluoro.

In an embodiment, the present invention relates to those compounds of Formula (I) and the pharmaceutically acceptable salts, and the solvates thereof, or any subgroup thereof as mentioned in any of the other embodiments, wherein R⁶, R⁷ and R⁸ represent hydrogen.

In an embodiment, the present invention relates to those compounds of Formula (I) and the pharmaceutically acceptable salts, and the solvates thereof, or any subgroup thereof as mentioned in any of the other embodiments, wherein R⁶, R⁷ and R⁸ represent fluoro.

In an embodiment, the present invention relates to those compounds of Formula (I) and the pharmaceutically acceptable salts, and the solvates thereof, or any subgroup thereof as mentioned in any of the other embodiments, wherein Het¹ is attached to the remainder of the molecule of Formula (I) through any available ring carbon atom.

In an embodiment, the present invention relates to those compounds of Formula (I) and the pharmaceutically acceptable salts, and the solvates thereof, or any subgroup thereof as mentioned in any of the other embodiments, wherein Het¹ is attached to the remainder of the molecule of Formula (I) through any available ring nitrogen atom.

In an embodiment, the present invention relates to those compounds of Formula (I) and the pharmaceutically acceptable salts, and the solvates thereof, or any subgroup thereof as mentioned in any of the other embodiments, wherein Het¹ is

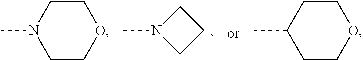

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het¹ is

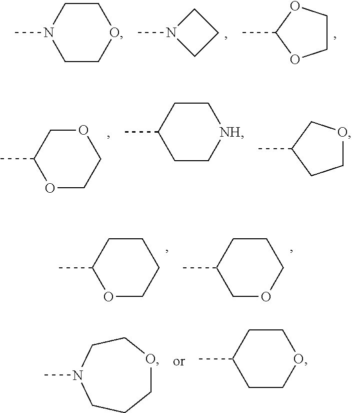

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Cy¹ is

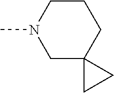

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het² is

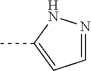

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het² is

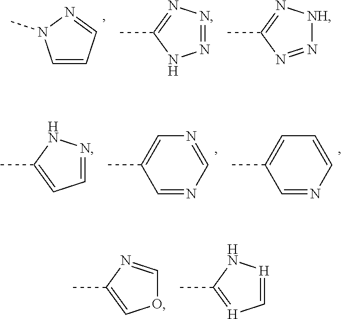

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het³ is

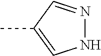

optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein Het² represents a 5- to 6-membered monocyclic aromatic ring containing one or two heteroatoms each independently selected from O, S, and N, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo.

In an embodiment, the present invention relates to those compounds of Formula (I) and the pharmaceutically acceptable salts, and the solvates thereof, or any subgroup thereof as mentioned in any of the other embodiments, wherein when R^{1a} and R^{1b} are taken together with the N-atom to which they are attached, together they form

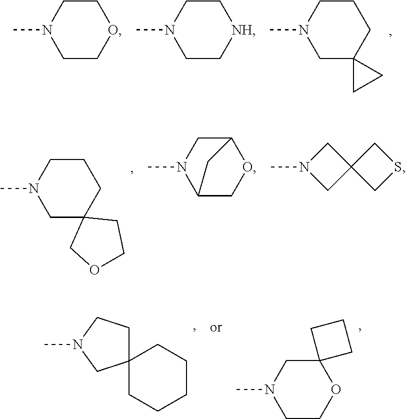

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein when R^{1a} and R^{1b} are taken together with the N-atom to which they are attached, together they form

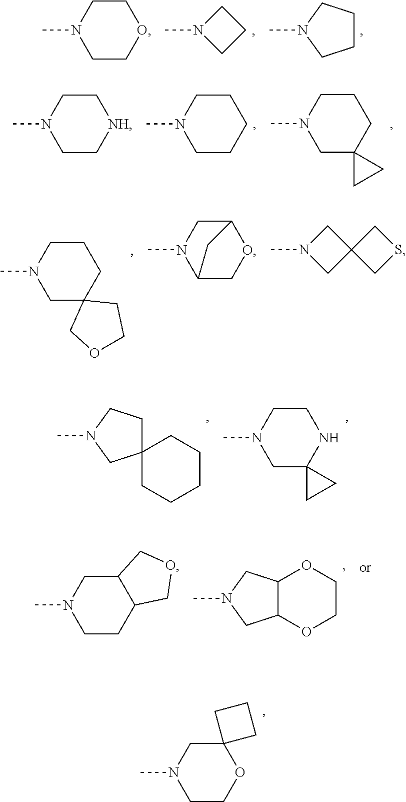

each optionally substituted according to any one of the other embodiments.

In an embodiment, the present invention relates to those compounds of Formula (I) and the pharmaceutically acceptable salts, and the solvates thereof, or any subgroup thereof as mentioned in any of the other embodiments, wherein when R^{d} and R^{e} are taken together with the N-atom to which they are attached, together they form 1-morholinyl.

In an embodiment, the present invention relates to those compounds of Formula (I) and the pharmaceutically acceptable salts, and the solvates thereof, or any subgroup thereof as mentioned in any of the other embodiments, wherein Het² is attached to the remainder of the molecule of Formula (I) through any available ring carbon atom.

In an embodiment, the present invention relates to those compounds of Formula (I) and the pharmaceutically acceptable salts, and the solvates thereof, or any subgroup thereof as mentioned in any of the other embodiments, wherein Het² is attached to the remainder of the molecule of Formula (I) through any available ring nitrogen atom.

In an embodiment, the present invention relates to those compounds of Formula (I) and the pharmaceutically acceptable salts, and the solvates thereof, or any subgroup thereof as mentioned in any of the other embodiments, wherein Cy¹ is attached to the remainder of the molecule of Formula (I) through any available ring carbon atom.

In an embodiment, the present invention relates to those compounds of Formula (I) and the pharmaceutically acceptable salts, and the solvates thereof, or any subgroup thereof as mentioned in any of the other embodiments, wherein Cy¹ is attached to the remainder of the molecule of Formula (I) through any available ring nitrogen atom.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-1):

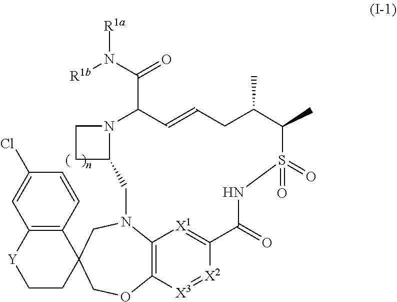

It will be clear that all variables in the structure of Formula (I-1), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-a′):

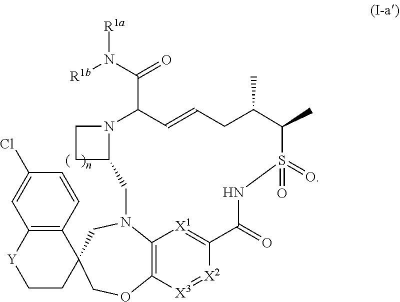

It will be clear that all variables in the structure of Formula (I-a′), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-a1):

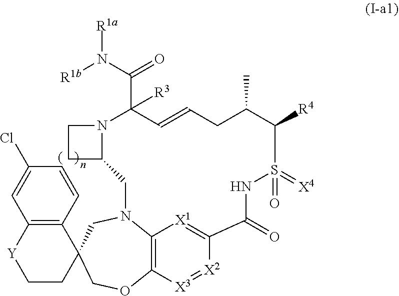

It will be clear that all variables in the structure of Formula (I-a1), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-b′):

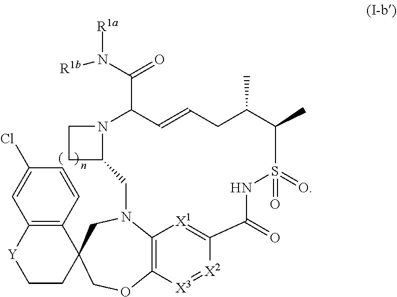

It will be clear that all variables in the structure of Formula (I-b′), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-b1):

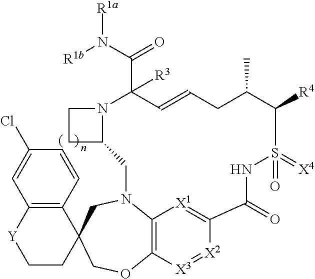

It will be clear that all variables in the structure of Formula (I-b1), are defined as defined for the compounds of Formula (I) or any subgroup thereof as mentioned in any of the other embodiments.

In an embodiment, the present invention relates to a subgroup of Formula (I) as defined in the general reaction schemes.

In an embodiment the compound of Formula (I) is selected from the group consisting of any of the exemplified compounds, and the free bases, the pharmaceutically acceptable salts, and the solvates thereof.

All possible combinations of the above indicated embodiments are considered to be embraced within the scope of the invention.

### Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or can be prepared by published methods. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art including also analogous reaction protocols as described in WO2016033486, WO2017147410 and WO2018183418.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

The meaning of the chemical abbreviations used in the schemes below are as defined in the schemes or as defined in Table 1.

The general schemes below focus on compounds of Formula (I-a1) and subgroups thereof, but a skilled person will understand that compounds of Formula (I-b1) can be synthesized by using analogous reaction procedures:

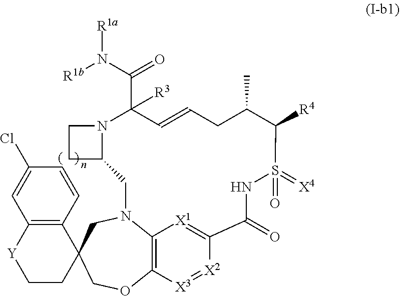

Compounds of Formula (I-a) wherein the variables are defined as in Formula (I) can be prepared according to Scheme 1,

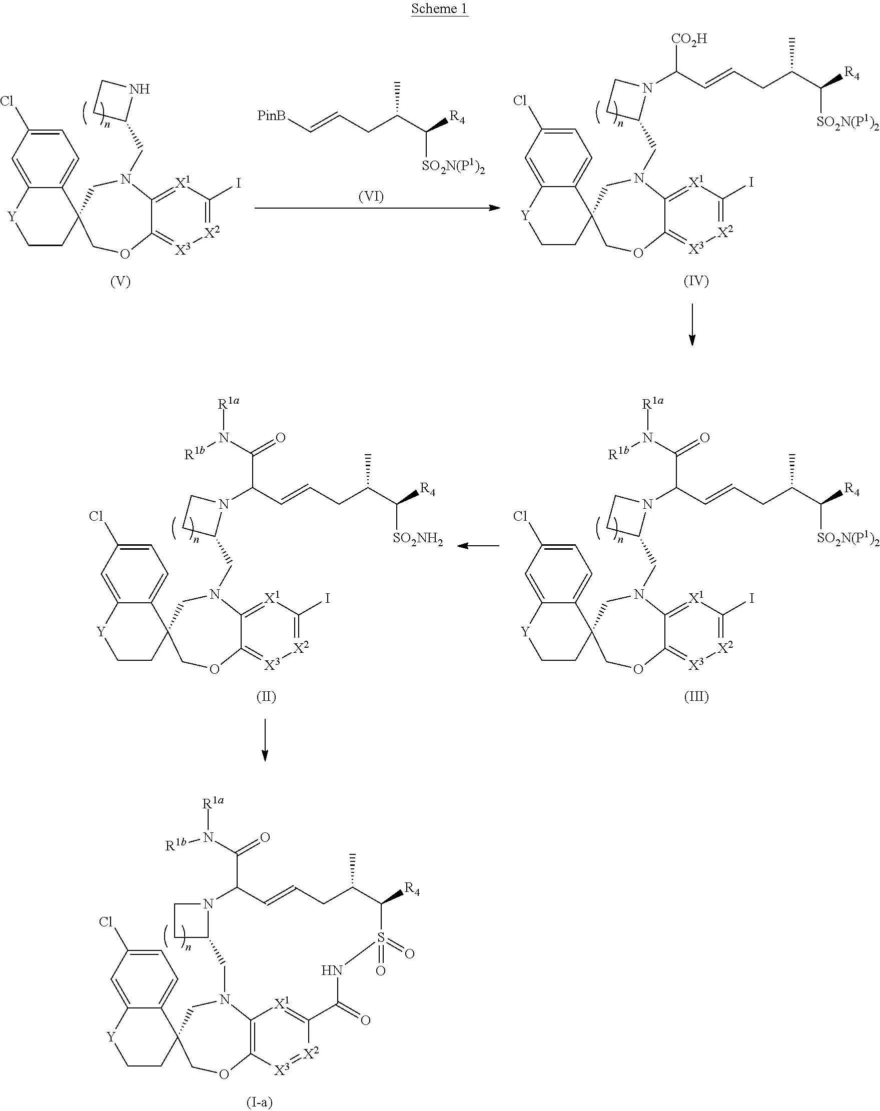

By reacting an Intermediate of Formula (II) in the presence of a suitable palladium catalyst such as, for example, [1,1′-bis(diphenylphosphino)ferrocene]palladium(II) dichloride, in a suitable solvent such as tetrahydrofuran, at a suitable temperature such as, for example, 80 or 100° C. and at a suitable CO pressure such as 30 bars.
Intermediates of Formula (II) can be prepared by deprotection of an Intermediate of Formula (III) in which P¹ is a suitable protective group such as, for example, para-methoxybenzyl (PMB), with a suitable deprotecting agent such as, for example, trifluoroacetic acid, in a suitable solvent such as, for example, dichloromethane at a suitable temperature such as, for example, room temperature.
Intermediates of Formula (III) can be prepared by reacting an Intermediate of Formula (IV) with an amine in presence of a suitable coupling agent such as, for example, propanephosphonic acid anhydride, a suitable base such as, for example, triethylamine, in a suitable solvent such as, for example, dichloromethane or ethyl acetate, at a suitable temperature such as, for instance, room temperature or 40° C.
Intermediates of Formula (IV) can be prepared by reacting an Intermediate of Formula (V) with an Intermediate of Formula (VI) (‘Pin’ means pinacol ester) and glyoxylic acid in a suitable solvent such as, for example, methanol, at a suitable temperature such as, for example, 40 or 60° C.

Compounds of Formula (I-a) where R⁴═H can alternatively be prepared according to Scheme 2,

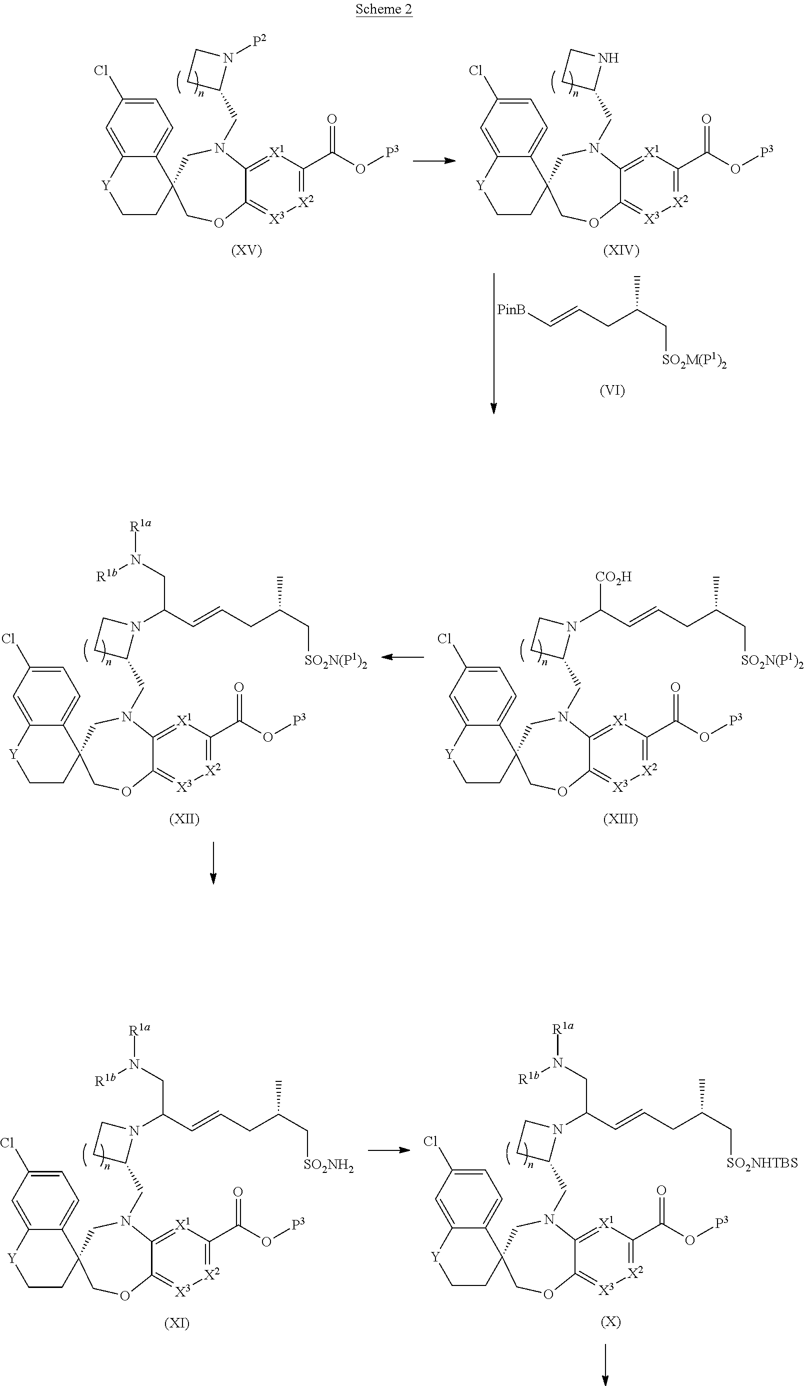

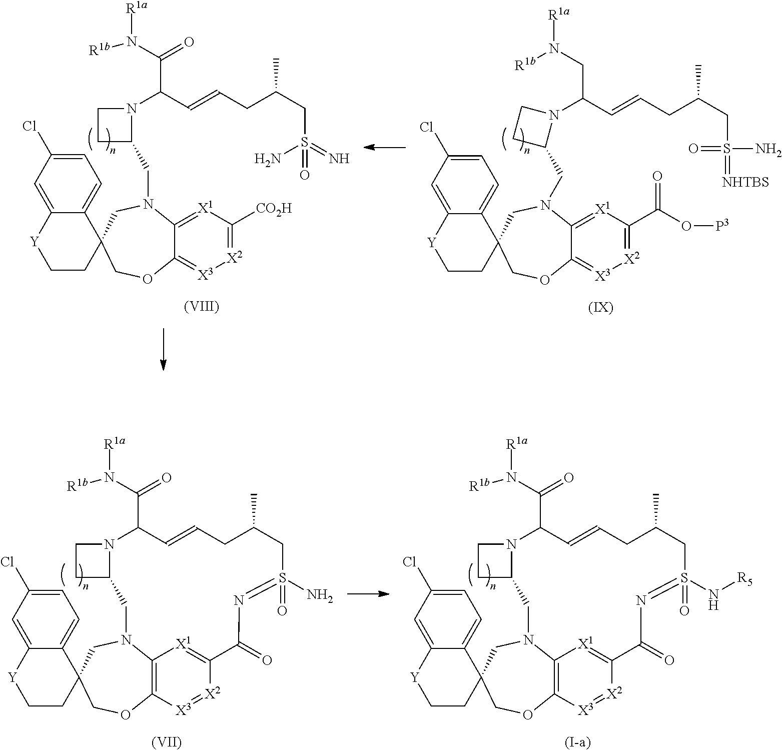

By reacting an Intermediate of Formula (VII) with a carboxylic acid in a presence of a suitable amide formation reagent such as, for example, HATU, HBTU, DCC or T3P, in presence of a base such as, for example, triethylamine, in a suitable solvent such as, for example, DMF, at a suitable temperature such as room temperature.
Intermediates of Formula (VII) can be prepared by reacting an Intermediate of Formula (VIII) with a suitable amide formation reagent such as, for example, diethyl cyanophosphonate, in a suitable solvent such as, for example, DMF, at a suitable temperature such as room temperature.
Intermediates of Formula (VIII) can be prepared by saponifying an Intermediate of Formula (IX) in which P³ is a suitable protective group such as, for instance, methyl, in the presence of a suitable base such as, for example, lithium hydroxide, in a suitable solvent such as, for example, tetrahydrofuran or methanol or water or a mixture thereof, at a suitable temperature such as room temperature or 50° C.
Intermediates of Formula (IX) can be prepared by reacting an Intermediate of Formula (X) with a suitable chlorinating agent such as, for example, chlorodiphenylphosphine, followed by treatment with a source of amine such as, for example, ammonia gas, in a suitable solvent such as, for example, THF, at a suitable temperature such as 0° C.
Intermediates of Formula (X) can be prepared by protection of an Intermediate of Formula (XI) with TBSCI (‘TBS’ means tert-butyldimethylsilyl) in the presence of a suitable base such as, for example, triethylamine, in a suitable solvent such as, for example, dichloromethane.
Intermediates of Formula (XI) can be prepared by deprotection of an Intermediate of Formula (XII) in which P¹ is a suitable protective group such as, for example, para-methoxybenzyl (PMB), with a suitable deprotecting agent such as, for example, trifluoroacetic acid, in a suitable solvent such as, for example, dichloromethane at a suitable temperature such as, for example, room temperature.
Intermediates of Formula (XII) can be prepared by reacting an Intermediate of Formula (XIII) with an amine using a suitable coupling agent such as, for example, propanephosphonic acid anhydride, in presence of a suitable base such as, for example, triethylamine, in a suitable solvent such as, for example, dichloromethane or ethyl acetate, at a suitable temperature such as, for instance, room temperature or 40° C.
Intermediates of Formula (XIII) can be prepared by reacting an Intermediate of Formula (XIV) with an Intermediate of Formula (VI) (‘Pin’ means pinacol ester) and glyoxylic acid in a suitable solvent such as, for example, methanol, at a suitable temperature such as, for example, 40 or 60° C.
Intermediates of Formula (XIV) can be prepared by reacting an Intermediate of Formula (XV) in which P² is a suitable protective group such as, for example, Boc, with a suitable deprotecting agent such as, for example, trifluoroacetic acid, as a suitable temperature such as, for example, room temperature.

Intermediates of Formula (V) and (XV) wherein X¹, X², X³, Y and n are as defined in Formula (I), wherein P² is a suitable protective group such as, for example, Boc, and wherein P³ is as defined in Scheme 2, can be prepared according to Scheme 3,

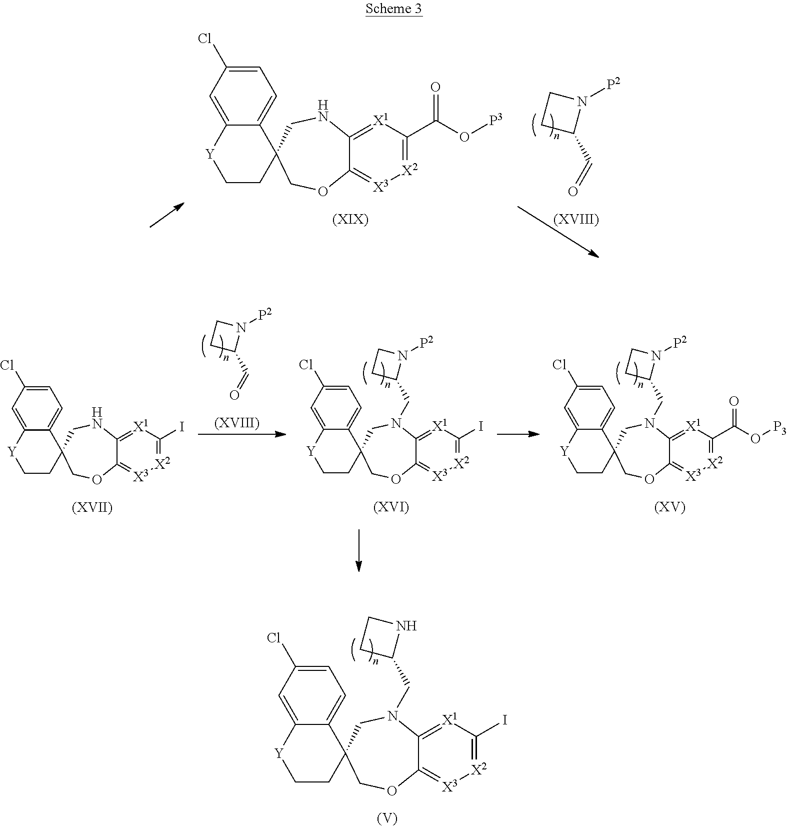

By reacting an Intermediate of Formula (XVI) in a CO insertion reaction with a suitable alcohol P³OH such as, for example, methanol, in the presence of a suitable palladium catalyst such as, for example, [1,1′-bis(diphenylphosphino)ferrocene]palladium(II) dichloride, in a suitable solvent such as tetrahydrofuran, at a suitable temperature such as 80 or 100° C. and at a suitable CO pressure such as 30 bar.
Alternatively, intermediates of Formula (XV) can be prepared by reacting an Intermediate of Formula (XIX) with an Intermediate of Formula (XVIII) in presence of a suitable reducing agent such as, for example, sodium cyanoborohydride or sodium triacetoxy borohydride, in a suitable solvent such as, for example, dichloromethane or acetic acid or a mixture thereof, at a suitable temperature such as, for example, 0° C. or room temperature.
Intermediates of Formula (XIX) can be prepared by reacting an Intermediate of Formula (XVII) in a CO insertion reaction with a suitable alcohol P³OH such as, for example, methanol, in the presence of a suitable palladium catalyst such as, for example, [1,1′-bis(diphenylphosphino)ferrocene]palladium(II) dichloride, in a suitable solvent such as tetrahydrofuran, at a suitable temperature such as 80 or 100° C. and at a suitable CO pressure such as 30 bar.
Intermediates of Formula (V) can be prepared by reacting an Intermediate of Formula (XVI) in which P² is a suitable protective group such as, for example, Boc, with a suitable deprotecting agent such as, for example, trifluoroacetic acid, at a suitable temperature such as, for example, room temperature.
Intermediates of Formula (XVI) can be prepared by reacting an Intermediate of Formula (XVII) with an Intermediate of Formula (XVIII) in presence of a suitable reducing agent such as, for example, sodium cyanoborohydride or sodium triacetoxy borohydride, in a suitable solvent such as, for example, dichloromethane or acetic acid or a mixture thereof, at a suitable temperature such as, for example, 0° C. or room temperature.

The synthesis of XVIII in which P² is Boc corresponds with (CAS [200184-45-8]) for n=1, (CAS [69610-41-9]) for n=2.

Intermediates of Formula (IX) wherein X¹, X², X³ are CH, and Y is as defined in Formula (I), can be prepared according to Scheme 4,

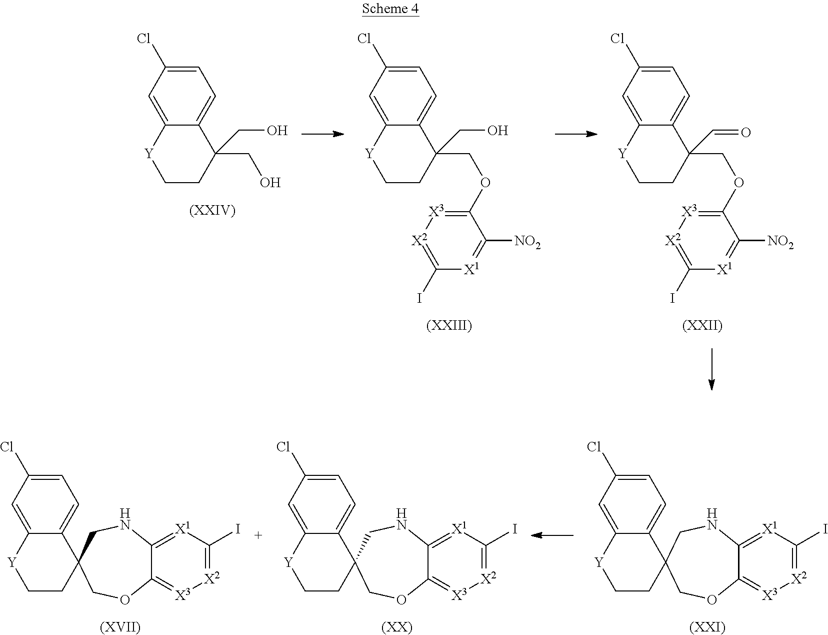

By separating the enantiomers of Formula (XXI) using an appropriate separation technique such as, for example, chiral SFC.
Intermediates of Formula (XXI) can be obtained by reacting an Intermediate of Formula (XXII) with a suitable reducing agent like, for instance, iron dust, in a suitable solvent such as, for instance, acetic acid, at a suitable temperature like, for instance, 70° C. It will be clear to a skilled person that the resulting aniline can condense intramolecularly with the aldehyde to an imine, which can be further reduced with a suitable reducing agent such as, for instance, sodium triacetoxyborohydride (NaBH(OAc)₃), in a suitable solvent such as, for instance, dichloromethane (DCM), at a suitable temperature such as, for instance, room temperature.
Intermediates of Formula (XXII) can be prepared by reacting an Intermediate of Formula (XXIII) with a suitable oxidant such as, for instance, dimethylsulfoxide (DMSO) and oxalyl chloride, in the presence of a suitable base such as, for instance, triethylamine, in a suitable solvent such as, for instance, DCM, at a suitable temperature such as, for instance, −78° C. or room temperature (rt).
Intermediates of Formula (XXIII) can be prepared by reacting an Intermediate of Formula (XXIV) with 1-fluoro-4-iodo-2-nitrobenzene (CAS [364-75-0]) in presence of a suitable base such as, for instance, K₂CO₃, in a suitable solvent such as, for instance, acetonitrile, at a suitable temperature such as, for instance, 50° C.

Intermediates of Formula (XV) can be prepared according to Scheme 5,

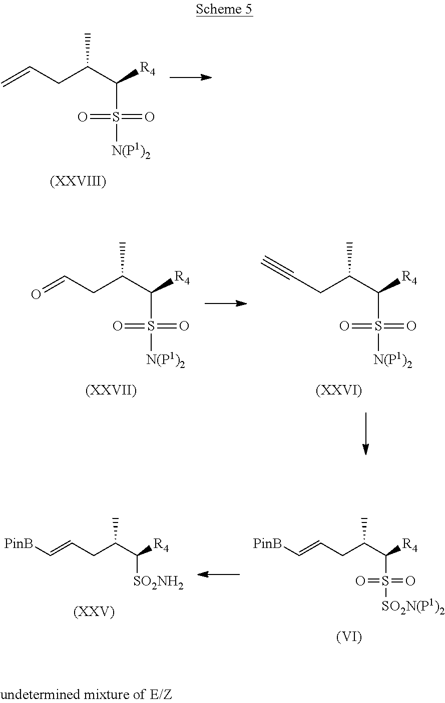

By deprotecting an Intermediate of Formula (VI) wherein P¹ is a suitable protective group such as, for example, p-methoxybenzyl, with an appropriate deprotecting agent such as, for example, trifluoroacetic acid, in a suitable solvent such as, for example, dichloromethane, at a suitable temperature such as, for example, 0° C. or room temperature.
Intermediates of Formula (VI) can be prepared by reacting Intermediate of Formula (XXVI) with bis(pinacolato)diboron, in the presence of a suitable additive such as, for example, triphenylphosphine, in the presence of a suitable base such as, for example, potassium phosphate dibasic, in the presence of a suitable catalyst such as, for example, copper (I) oxide, in a suitable solvent such as, for example, methanol, at a suitable temperature such as, for example, room temperature.
Intermediates of Formula (XXVI) can be prepared by reacting an Intermediate of Formula (XXVII) with dimethyl (1-diazo-2-oxopropyl)phosphonate, in the presence of a suitable base, for example, potassium carbonate, in a suitable solvent such as, for example, methanol, at a suitable temperature such as, for example, 0° C.
Intermediates of Formula (XXVII) can be prepared either by ozonolysis of an Intermediate of Formula (XXVIII) in a suitable solvent such as, for example, dichloromethane or methanol, at a suitable temperature such as, for example, −78° C., or by oxidation of an Intermediate of Formula (XXVIII) with suitable reagents such as, for example, a catalytic amount of osmium tetroxide with sodium periodate, in a suitable solvent such as, for example, tetrahydrofuran and water mixture, at a suitable temperature such as, for example, room temperature.

(XXVIII) in which P¹ is p-methoxybenzyl and R⁴ is methyl corresponds with (CAS [1883727-77-2]). (XXVIII) in which P¹ is p-methoxybenzyl and R⁴ is hydrogen corresponds with (CAS [1883727-89-6]).

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (Boc), benzyloxycarbonyl (CBz) and 9-fluorenylmethyleneoxycarbonyl (Fmoc). The need for such protection is readily determined by one skilled in the art. For a general description of protecting groups and their use, see T. W. Greene and P. G. M. Wuts, Protective Groups in Organic Synthesis, 4th ed., Wiley, Hoboken, New Jersey, 2007.

### Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (IES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8: Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### Examples

Several methods for preparing the Compounds of this invention are illustrated in the following examples. Unless otherwise noted, all starting materials were obtained from commercial suppliers and used without further purification, or alternatively can be synthesized by a skilled person by using published methods.

TABLE 1
Abbreviations

| Abbreviation | Meaning |
|---|---|
| AcOH | acetic acid |
| Boc | tert-butyloxycarbonyl |
| Celite ® | diatomaceous earth |
| DCE | 1,2-dichloroethane |
| DCM | dichloromethane |
| Dess-Martin | 1,1,1-tris(acetyloxy)-1,1-dihydro- |
| periodinane | 1,2-benziodoxol-3-(1H)-one |
| DEAD | diethyl azodicarboxylate |
| dicalite ® | diatomaceous earth |
| DIPE | diisopropyl ether |
| DMF | N,N-dimethylformamide |
| DMSO | dimethyl sulfoxide |
| DMT | dimercaptotriazine |
| eq | equivalent(s) |
| Et | ethyl |
| Et₃N or TEA | triethylamine |
| EtOAc or AcOEt | ethyl acetate |
| EtOH | ethanol |
| g | gram(s) |
| | hour(s) |
| HBTU | 2-(1H-benzotriazole-1-yl)-1,1,3,3- |
| | tetramethyluronium hexafluorophosphate |
| HPLC | high performance liquid chromatography |
| iPrNH₂ | isopropylamine |
| LiHMDS | lithium bis(trimethylsilyl)amide |
| Me | methyl |
| MeOH | methanol |
| min | minute(s) |
| NaBH(OAc)₃ | sodium triacetoxyborohydride |
| NaOAc | sodium acetate |
| NH₄OAc | ammonium acetate |
| PBu₃ | tributylphosphine |
| PMB | p-methoxybenzyl |
| PPh₃ or Ph₃P | triphenylphosphine |
| Prep | preparative |
| RM | reaction mixture |
| RP | reversed phase |
| RT | room temperature |
| Rt or Rt | retention time (in minutes) |
| SFC | supercritical fluid chromatography |
| SiliaMetS ® DMT | SiliaMetS DMT (dimercaptotriazine) |
| | is the silica-bound equivalent of |
| | 2,4,6-trimercaptotriazine |
| | (trithiocyanuric acid, TMT). It is |
| | a versatile metal scavenger. |
| TFA | trifluoroacetic acid |
| THF | tetrahydrofuran |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

In case stereobonds are shown in the structures of the intermediates and compounds of this invention, this means that the stereochemistry is absolute and determined, irrespective of the fact if stereodescriptors were also added or not.

### Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

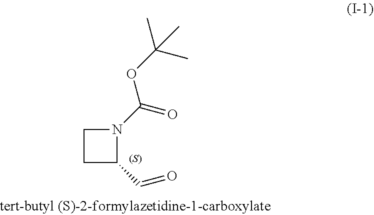

Dess-Martin periodinane [87413-09-0](30 g, 1.3 eq.) was added to a stirred solution of (S)-1-Boc-2-azetidinemethanol [161511-85-9](10 g, 53.4 mmol) in DCM (250 mL) at 0° C. The reaction mixture was stirred at room temperature for 2 h. The reaction mixture was quenched by the addition of a solution of sodium thiosulfate in a saturated aqueous NaHCO₃ solution. The resulting mixture was stirred vigorously for 15 min. The resulting suspension was filtered over a pad of Celite®. The filter pad was washed with dichloromethane. The combined filtrate was separated, and the aqueous layer was extracted with DCM (2×). The combined organic layer was washed twice with saturated aqueous NaHCO₃, dried with MgSO₄, and filtered. The solvents of the filtrate were evaporated to give 10.17 g of Intermediate 1 (assumed quant. yield) as an oil, used without further purification.

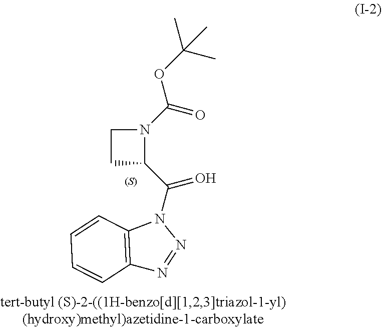

Tert-butyl methyl ether (40 mL) was added to I-1 (9 g, 48.59 mmol). The resulting suspension was stirred for 10 minutes at room temperature and then filtered. The solid was rinsed with tert-butyl methyl ether. The filtrate was transferred to a round bottom flask and then cooled down to 0° C. 1H-benzotriazole (5.79 g, 1 eq.) was added to the solution and the reaction mixture was stirred at room temperature for 18 h. The solvents were evaporated to give Intermediate 2 (14.79 g, assumed quant. yield), used without further purification.

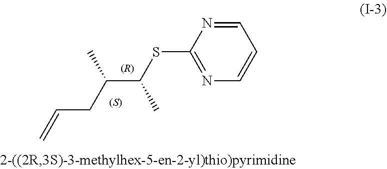

A solution of DEAD (25.79 mL, 1.7 eq.) in THE (100 mL) was added dropwise to PBu₃ (36.09 mL, 1.5 eq.) in degassed THE (300 mL) under nitrogen atmosphere at 0° C. A solution of (2S,3S)-3-methylhex-5-en-2-ol [125225-80-1](11 g, 1 eq.) in THE (200 mL) was added dropwise to the mixture at 0° C. The mixture was stirred at 0° C. for 30 min (the solution turned light orange). Pyrimidine-2-thiol [131242-36-9](30.79 g, 2.85 eq.) was added gradually to the mixture. The reaction mixture was stirred at 0° C. for 1 h and then at room temperature overnight. The reaction mixture was filtered. To the filtrate was added 500 mL EtOAc. The solution was washed twice with 1 N K₂CO₃ (300 mL) and then twice with brine (300 mL). The aqueous layer was back-extracted with 400 mL EtOAc. The combined organic layer was dried over Na₂SO₄ and concentrated in vacuo. The residue was purified by flash column chromatography over silica gel (eluent: petroleum ether/EtOAc from 100/0 to 0/100). The desired fractions were collected and the solvent was concentrated to dryness under vacuum to give Intermediate 3 (35.4 g, 68% yield).

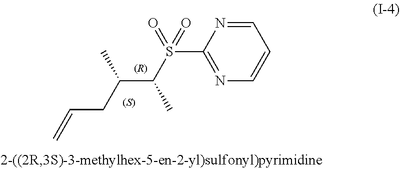

To a mixture of Na₂WO₄ [10213-10-2](1.075 g, 0.1 eq.), phenylphosphonic acid [157171-33-1](0.515 g, 0.1 eq.) and tetrabutylammonium sulfate (3.75 mL, 0.1 eq.) was added H₂O₂ (9.24 g, 2.5 eq.) all at once at room temperature. The mixture was aged at room temperature for 5 minutes before a solution of I-3 (10 g, 1 eq.) in toluene (150 mL) was added in one portion. The biphasic reaction mixture was heated to 50° C. with vigorous stirring. After 60 minutes the reaction was cooled to room temperature. The reaction mixture was poured into water (150 mL). The layers were separated and the aqueous layer was extracted with EtOAc (300 mL×3). The combined organic layer was washed with a saturated aqueous solution of Na₂S₂O₅ (200 mL×2), dried over MgSO₄, filtered, and concentrated. The residue was purified by flash column chromatography over silica gel (eluent: petroleum ether/EtOAc from 100/0 to 0/100). The desired fractions were collected and the solvent was concentrated under vacuum to give Intermediate 4 (5.6 g, 68%) as a yellow oil.

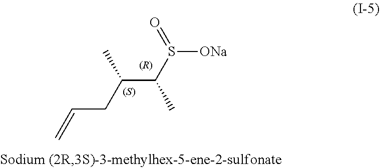

A solution of I-4 (5.6 g, 1 eq.) in MeOH (30 mL) was cooled to 0° C. and treated with NaOMe (4.794 g, 1 eq.). The mixture was allowed to warm to room temperature for 20 min. The solvent was removed under vacuum. Water (10 mL) was added to the mixture and it was extracted with EtOAc (10 mL×3). The combined organic layer was concentrated under vacuum to give Intermediate 5 (6.3 g, assumed quant. yield) as a yellow solid. The product was used for the next step without further purification.

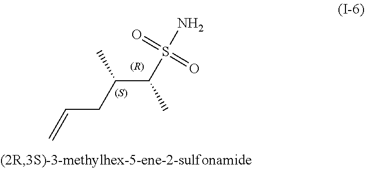

I-5 (33.2 g, 1 eq.) was dissolved in MeOH (200 mL) and treated with NaOAc (18.48 g, 1.25 eq.), followed by a solution of hydroxylamine O-sulfonic acid (25.48 g, 1.25 eq.) in water (15 mL). The reaction was stirred overnight at room temperature. The mixture was neutralized with solid NaHCO₃ and extracted with EtOAc (400 mL×3). The combined organic layers was dried over MgSO₄, filtered and concentrated. The residue was purified by flash column chromatography over silica gel (eluent: petroleum ether/EtOAc from 100/0 to 0/100). The desired fractions were collected, and the solvent was concentrated under vacuum to give Intermediate 6 (18.1 g, 56%) as clear oil.

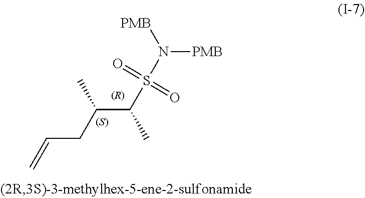

To a solution of I-6 (5 g, 28.2 mmol) in DMF (50 mL) was added K₂CO₃ (15.593 g, 4 eq.), followed by slow addition of 4-methoxybenzyl chloride (11.474 mL, 3 eq). Once the addition was complete, the reaction was heated to 70° C. and was stirred at this temperature overnight. The reaction was filtered through a pad of Dicalite® to remove the inorganics and concentrated under reduced pressure to give a pale-yellow oil. The oil was dissolved in EtOAc (150 mL) and washed with brine (2×100 mL). The organic layer was dried over MgSO₄, filtered, and concentrated under reduced pressure to afford a pale-yellow oil. The crude product was purified by flash column chromatography on silica gel (heptane:EtOAc—1:0 to 8:2). After evaporation of the fractions containing product, the residue was purified by preparative HPLC (Stationary phase: RP XBridge Prep C18 OBD—10 μm, 50×150 mm, mobile phase: 0.25% NH₄HCO₃ solution in water, CH₃CN) to give Intermediate 7 (5.98 g, yield 48%) as a white solid.

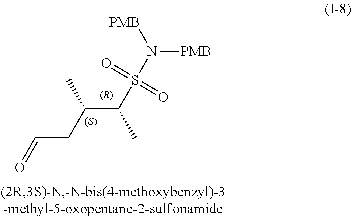

I-7 (1 g, 2.275 mmol) was dissolved in a mixture of DCM (12.5 mL) and MeOH (12.5 mL) and the resulting mixture was cooled to −78° C. Ozone (109.199 mg, 1 eq.) was subsequently bubbled through the reaction mixture until a blue persistent color was observed (5 min). Nitrogen was then bubbled through the solution (still at −78° C.) to remove the blue color and this was followed by addition of PPh₃ (2.984 g, 5 eq). Once the addition was complete, the reaction was left stirring at −78° C. for 1 h. The reaction mixture was then slowly allowed to warm to room temperature and was stirred for 1 h. The heterogenous mixture was filtered through a pad of Dicalite®. The pad was thoroughly washed with DCM. The filtrate was concentrated under reduced pressure to give a green oil that was purified by flash column chromatography on silica gel (heptane:EtOAc—1:0 to 3:1) to give Intermediate 8 (920 mg, yield 87%) as a colorless oil.

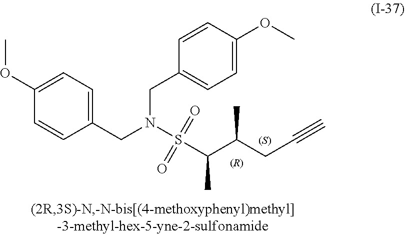

Dimethyl (1-diazo-2-oxopropyl)phosphonate (0.3 mL, 1 eq.) was added to a suspension of I-8 (800 mg, 1.9 mmol) and K₂CO₃ (527 mg, 2 eq.) in MeOH (5 mL) at 0° C. After 30 min at 0° C., the reaction mixture was allowed to reach room temperature and stirring was continued for 4 h. The reaction mixture was diluted with 10 mL DCM, filtered, and the filtrate was evaporated. The residue was purified via preparative HPLC (Stationary phase: RP XBridge Prep C18 OBD—10 μm, 50×150 mm, mobile phase: 0.25% NH₄HCO₃ solution in water, CH₃CN), yielding Intermediate 37 (500 mg, yield: 63%).

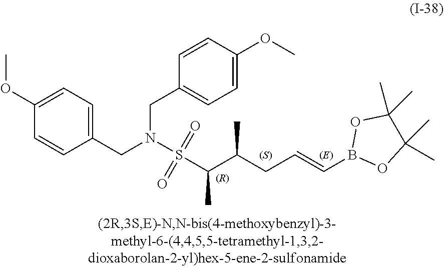

A mixture of bis(pinacolato)diboron (187 mg, 1.5 eq.), copper(I) oxide (19.4 mg, 0.27 eq.), PPh₃ (49 mg, 0.38 eq.), potassium phosphate dibasic (171 mg, 2 eq.) and MeOH (1.6 mL) was stirred in a tube. The tube was closed and purged with nitrogen for 10 min. I-37 (200 mg, 0.48 mmol) dissolved in MeOH (1.6 mL) was added. The reaction mixture was stirred at room temperature for 3 h. EtOAc (6 mL) was added. The reaction mixture was stirred for 5 min and was filtered. The filtrate was evaporated and the residue was stirred in DIPE, the solid was filtered off, and the filtrate was evaporated, yielding Intermediate 38 (243 mg, yield: 93%), used without further purification.

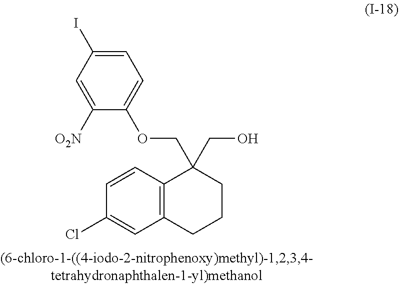 [SMILES: O=[N+]([O-])c1cc(I)ccc1OCC1(CO)CCCc2cc(Cl)ccc21]

This reaction was performed in two batches. For each batch, 6-chloro-1,2,3,4-tetrahydronaphthalene-1,1-diyl)dimethanol (CAS [1883726-74-6], 300 g, 1.32 mol) and 1-fluoro-4-iodo-2-nitrobenzene (353 g, 1.32 mol) were dissolved in acetonitrile (1.4 L). K₂CO₃ (549 g, 3.97 mol) was added to the reaction mixture and it was stirred at 50° C. for 16 h. The reaction mixture was filtered, and the filtrate was evaporated. The two batches were then combined and the residue was purified by column chromatography (SiO₂, petroleum ether:dichloromethane=3/1 to petroleum ether/EtOAc=1:1). Intermediate 18 was obtained as a yellow oil (600 g, 48% yield).

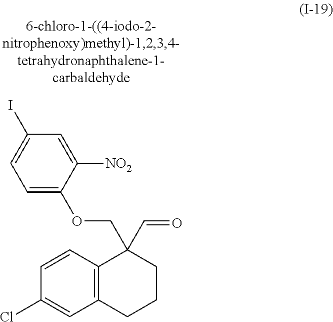 [SMILES: O=CC1(COc2ccc(I)cc2[N+](=O)[O-])CCCc2cc(Cl)ccc21]

This reaction was performed in three batches. For each batch, DMSO (99.0 g, 1.27 mol) was added to a solution of (COCl)₂ (161 g, 1.27 mol) in DCM (2.4 L) at −78° C. The reaction mixture was stirred at −78° C. for 15 min. I-18 (200 g, 422 mmol) in DCM (0.90 L) was then added at −78° C. and stirring was continued for 30 min. at −78° C. Et₃N (214 g, 2.11 mol) was added at −78° C. and the reaction mixture was allowed to warm to room temperature. Stirring was continued at room temperature for 1.5 h. Aqueous NaHCO₃ (1 L) was added and the mixture was extracted with DCM (0.5 L×2). The three batches were then combined and evaporated to yield Intermediate 19 (560 g) as a yellow solid, used without further purification.

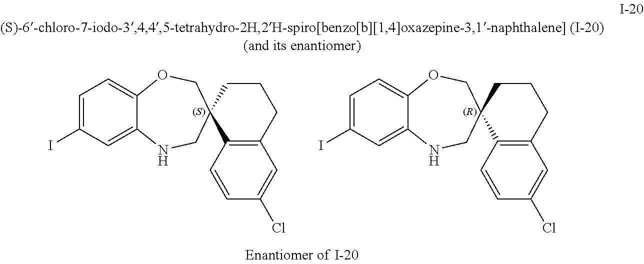

This reaction was performed in three batches. Iron (153 g, 2.75 mol) was added to a solution of I-19 (185 g, 392 mmol) in AcOH (2.5 L) at 70° C. and the reaction mixture was stirred at 70° C. for 3 h. The solvent was evaporated and DCE (1.9 L) was added to the residue. NaBH(OAc)₃ (333 g, 1.57 mol) was then added portionwise at 0° C. Stirring was continued at room temperature for 1 h. The three batches were combined. Citric acid (10% solution in water, 5. L) was added and the mixture was extracted with DCM (2 L ×2). The combined organic layer was evaporated. The residue was purified by SFC (column: DAICEL CHIRALPAK AD (250×50 mm, 10 m); mobile phase: [0.1% NH₃H₂O in EtOH]; B %: 50%-50%, 8.5 min) to afford Intermediate 20 (95.2 g, 40% yield) and its enantiomer (105.1 g, 44% yield), both as yellow solids.

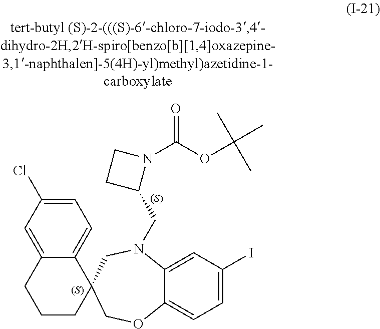

A mixture of I-20 (7.58 g, 17.8 mmol) and I-2 (16.25 g, 53.4 mmol) were dissolved in DCM (75 mL) and AcOH (25 mL) was added. The mixture was stirred for 30 minutes at room temperature and then cooled to 0° C. Sodium triacetoxyborohydride (11.3 g, 53.4 mmol) was added portionwise. After addition, the reaction mixture was stirred at room temperature for 16 hours. The reaction mixture was poured portionwise into a cooled (0° C.) solution of NaOH (21.3 g, 53.4 mmol) in 300 mL water. After addition the mixture was diluted with DCM and water. The organic layer was separated, washed with water, dried with MgSO₄, filtered, and the solvents of the filtrate were evaporated under reduced pressure. The residue was dissolved in DCM and purified by flash chromatography over silica (eluent: dichloromethane). The fractions containing product were combined and the solvents were evaporated yielding Intermediate 21 (9.8 g, yield 92%).

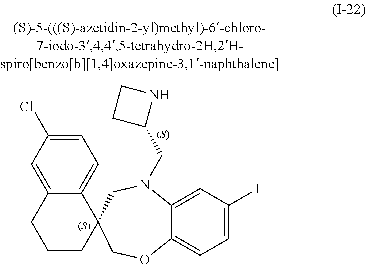

A mixture of I-21 (9.8 g, 16.5 mmol) and DCM (125 mL) was stirred at room temperature. TFA (125 ml) was added dropwise. The reaction mixture was stirred at room temperature for 3 hours and was then evaporated to dryness keeping the temperature below 30° C. The residue was taken up into DCM and was poured into an aqueous NaHCO₃ solution. The layers were separated and the organic layer was extracted with DCM. The combined organic fractions were washed with water, dried with MgSO₄, and evaporated. The residue was purified by flash chromatography on silica (eluent: DCM-MeOH/NH₃ gradient 100%/0% to 95%/5%) The pure fractions were collected and evaporated. The residual oil was stirred in CH₃CN until precipitation occurred. The precipitate was filtered off and dried yielding Intermediate 22 (5.1 g, yield 63%).

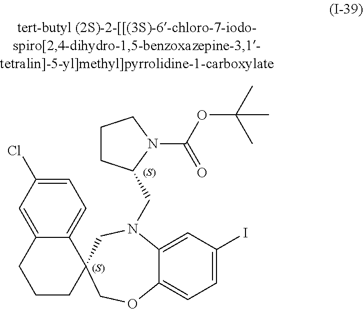

I-20 (13.197 g, 31 mmol) and N-Boc-L-prolinal (18.53 g, 3 eq.) were dissolved in CH₂Cl₂ (150 mL) and then AcOH (35.5 mL, 20 eq.) was added. The mixture was stirred for 30 min at room temperature and then cooled down to 0° C. Then sodium triacetoxyborohydride (19.711 g, 3 eq.) was added portionwise. After addition the reaction mixture was stirred at room temperature for 18 hours. The reaction mixture was poured out portionwise into a cooled (0° C.) solution of NaOH (31 g) in 620 mL water. After addition the mixture was diluted with CH₂Cl₂ and water. The organic layer was separated, washed with water, dried with MgSO₄, filtered and the solvents of the filtrate were evaporated. The residue was purified by flash chromatography on silica gel (eluent: CH₂Cl₂) The fractions containing product were combined and the solvents were evaporated. This residue was purified again by HPLC (gradient ethylacetate/hexane) to give Intermediate 39 (12.2 g, yield: 64%).

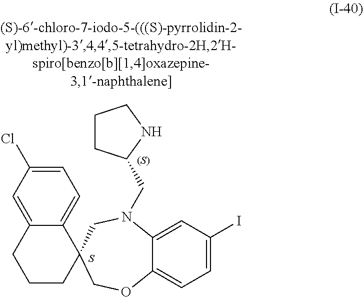

To a stirred solution of I-39 (14 g, 22.991 mmol) in anhydrous DCM (230 mL) was added TFA (23 mL, 1.49 g/mL, 300.551 mmol). The resulting mixture was stirred at room temperature for 20 h. The mixture was carefully quenched with saturated aqueous NaHCO₃ solution and DCM. The organic layer was extracted, washed with brine (100 mL), dried over Na₂SO₄, filtered off and the solvent evaporated under reduced pressure. The crude was used in the next step without further purification.

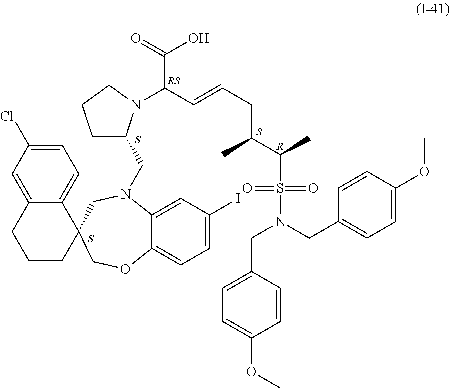

A solution of I-38 (684 mg, 1.26 mmol), I-40 (640.33 mg, 1.26 mmol) and glyoxylic acid monohydrate (231.68 mg, 2.52 mmol) in MeOH (40.27 mL) was stirred at 65° C. for 16 h. The solvent from the reaction mixture was evaporated under reduced pressure. The residue was purified by flash column chromatography on silica gel using as eluent gradient DCM/MeOH 95/5 to yield intermediate 41 (1025 mg, 83% yield).

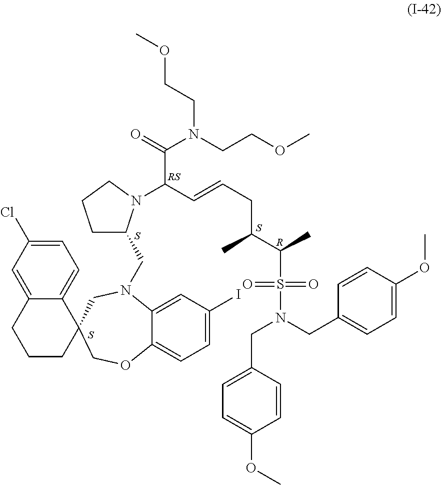

1-Propanephosphonic anhydride (454 μL, 0.38 mmol) was added dropwise to a stirred solution of 1-41 (125 mg, 0.127 mmol), bis(2-methoxyethyl)amine (50.84 mg, 0.382 mmol) and TEA (125.5 μL, 0.905 mmol) in DCM (3.75 mL) at 0° C. The reaction mixture was stirred for 2 h. The reaction mixture was diluted with dichloromethane (10 mL) and poured into water. The aqueous layer was extracted with DCM. The organic layer was dried with MgSO₄ and the solvent evaporated under reduced pressure. The residue was purified by flash column chromatography on silica gel using as eluent gradient DCM/MeOH 96/4 to give intermediate 42 (110 mg, 78% yield) as an oil.

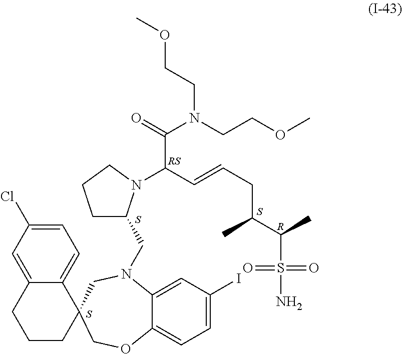

To a solution of I-42 (110 mg, 0.1 mmol) in DCM (3 mL) at 0° C. was added dropwise TFA (0.77 mL, 10 mmol). After the addition, the reaction mixture was warmed to 25° C. and stirred at this temperature for 16 h. The reaction mixture was diluted with DCM (30 mL) and was added dropwise into a cold mixture of NaHCO₃ (1.68 g, 20 mmol) and water (100 mL). The layers were separated, the aqueous layer was extracted with DCM (30 mL, ×1). The combined organic layers were dried over MgSO₄, filtered and the solvent removed under reduced pressure. The residue was purified by flash column chromatography on silica gel using as eluent gradient DCM/MeOH 95/5. The pure fractions were collected and the solvent evaporated under reduced pressure. The residue was co-evaporated with toluene (×2) to yield intermediate 43 (90 mg, assumed quant. yield) as an oil.

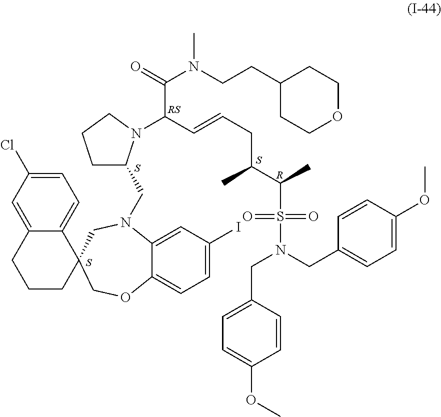

1-Propanephosphonic anhydride (757.36 μL, 0.64 mmol) was added dropwise to a stirred solution of I-41 (250 mg, 0.25 mmol), methyl[2-(oxan-4-yl)ethyl]amine (CAS [1083216-46-9]) (91.12 mg, 0.64 mmol) and TEA (251.01 μL, 1.81 mmol) in DCM (7.53 ml) at 0° C. The reaction mixture was stirred at room temperature for 2 h. The reaction mixture was diluted with dichloromethane and poured into water. The aqueous layer was extracted with DCM. The organic layer was dried with MgSO₄ and the solvent evaporated under reduced pressure. The residue was purified by flash column chromatography on silica gel using as eluent gradient DCM/MeOH 96/4 to give intermediate 44 (230 mg, 82% yield).

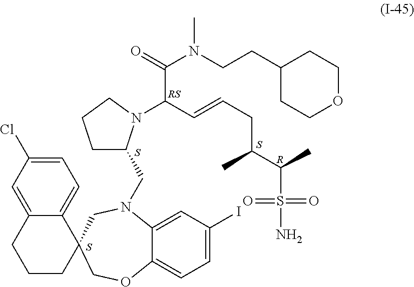

To a solution of I-44 (230 mg, 0.21 mmol) in DCM (3.25 mL) at 0° C. was added dropwise TFA (2.92 mL, 38.1 mmol). After the addition, the reaction mixture was warmed to 25° C. and stirred at this temperature for 16 h. The reaction mixture was diluted with DCM (30 mL) and was added dropwise into a cold mixture of NaHCO₃ (4268 mg, 50.8 mmol) and water (100 mL). The layers were separated, the aqueous layer was extracted with DCM (30 mL, ×1). The combined organic layers were dried over MgSO₄, filtered and the solvent removed under reduced pressure. The residue was purified by flash column chromatography on silica gel using as eluent gradient DCM/MeOH 95/5. The pure fractions were collected and the solvent evaporated under reduced pressure. The residue was co-evaporated with toluene (x 2) to yield intermediate 45 (195 mg, assumed quant. yield).

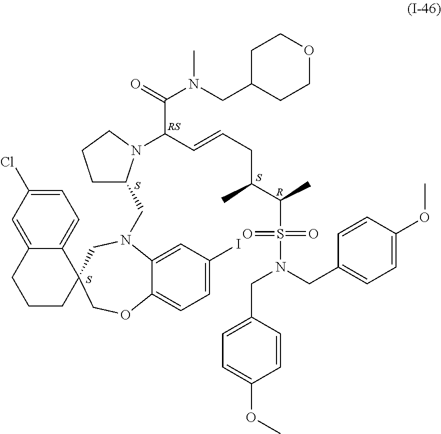

1-propanephosphonic anhydride (454.4 μL, 0.382 mmol) was added dropwise to a stirred solution of I-41 (125 mg, 0.127 mmol), TEA (125 μL, 0.91 mmol) and N-methyl(tetrahydro-2H-pyran-4-yl)methanamine (49 mg, 0.38 mmol) in DCM (3.75 ml) at 0° C. After the addition, the reaction mixture was stirred at room temperature for 2 hours. The reaction mixture was diluted with dichloromethane and poured into water. The aqueous layer was extracted with DCM. The organic layer was dried with MgSO₄ and the solvent evaporated. The residue was purified by flash column chromatography on silica gel using as eluent DCM/MeOH 96/4 to yield intermediate 46 (140 mg, yield assumed quant. yield).

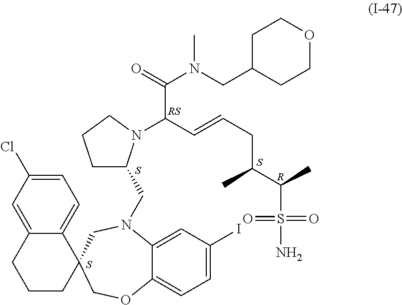

To a solution of I-46 (140 mg, 0.128 mmol) in DCM (3 mL) at 0° C. was added dropwise TFA (0.98 mL, 12.8 mmol). After the addition, the reaction mixture was stirred at 25° C. for 16 h. The reaction mixture was diluted with DCM (30 mL) and was added dropwise into a cold mixture of NaHCO₃ (2.15 g, 25.6 mmol) and water (100 mL). The organic layer was separated, the aqueous layer was extracted with DCM (x 1, 30 mL). The combined organic layers were dried over MgSO₄, filtered and the solvent evaporated under reduced pressure. The residue was purified by flash column chromatography on silica gel using as eluent gradient DCM/MeOH 95/5. The pure fractions were collected and evaporated. The residue was co-evaporated with toluene (x 2) to yield intermediate 47 (90 mg, 82% yield) as an oil.

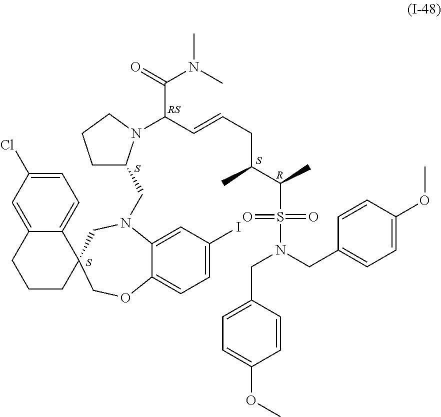

1-propanephosphonic anhydride (757.36 μL, 0.64 mmol) was added dropwise to a stirred solution of I-41 (250 mg, 0.25 mmol), TEA (251.01 μL, 1.81 mmol) and dimethyl amine (0.58 mL, 1.15 mmol) in DCM (7.53 ml) at 0° C. After the addition, the reaction mixture was stirred at room temperature for 16 hours. The reaction mixture was diluted with dichloromethane and poured into water. The aqueous layer was extracted with DCM. The organic layer was dried with MgSO₄ and the solvent evaporated. The residue was purified by flash column chromatography on silica gel using as eluent DCM/MeOH 97/3 to yield intermediate 48 (272 mg, assumed quant. yield).

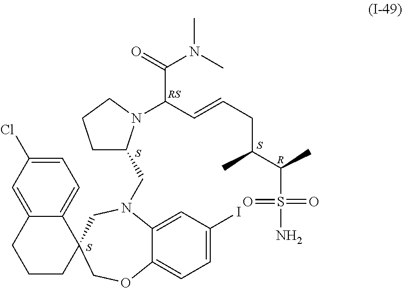

To a solution of I-48 (256.41 mg, 0.25 mmol) in DCM (3.25 mL) at 0° C. was added dropwise TFA (2.92 mL, 38.1 mmol). After the addition, the reaction mixture was stirred at 25° C. for 15 h. The reaction mixture was diluted with DCM (30 mL) and was added dropwise into a cold mixture of NaHCO₃ (4268 mg, 50.8 mmol) and water (100 mL). The organic layer was separated, the aqueous layer was extracted with DCM (30 mL, ×1). The combined organic layers were dried over MgSO₄, filtered and the solvent evaporated under reduced pressure. The residue was purified by flash column chromatography on silica gel using as eluent gradient DCM/MeOH 96/4. The pure fractions were collected and evaporated. The residue was co-evaporated with toluene (×2) to yield intermediate 49 (180 mg, 92% yield).

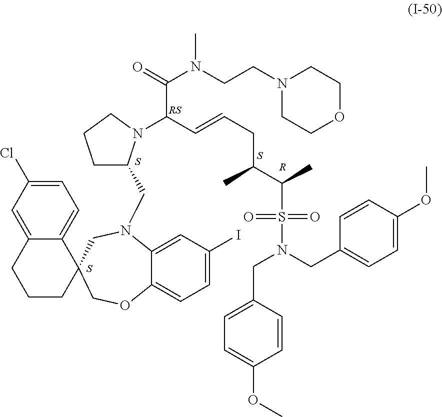

1-propanephosphonic anhydride (757.36 μL, 0.64 mmol) was added dropwise to a stirred solution of 1-41 (250 mg, 0.25 mmol), TEA (251.01 μL, 1.81 mmol) and N-methyl-2-morpholinoethanamine (91.75 mg, 0.64 mmol) in DCM (7.53 mL) at 0° C. After the addition, the reaction mixture was stirred at room temperature for 2 hours. The reaction mixture was diluted with dichloromethane and poured into water. The aqueous layer was extracted with DCM. The organic layer was dried with MgSO₄ and solvent evaporated. The residue was purified by flash column chromatography on silica gel using as eluent DCM/MeOH 96/4 to yield intermediate 50 (304 mg, assumed quant. yield).

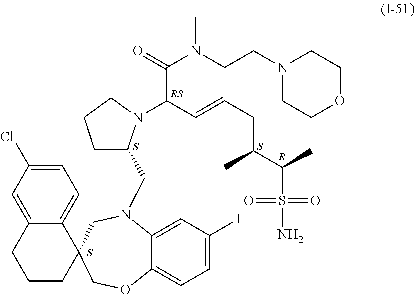

To a solution of 1-50 (281.59 mg, 0.25 mmol) in DCM (3.25 mL) at 0° C. was added dropwise TFA (2.92 mL, 38.1 mmol). After the addition, the reaction mixture was stirred at 25° C. for 16 h. The reaction mixture was diluted with DCM (30 mL) and was added dropwise into a cold mixture of NaHCO₃ (4268 mg, 50.8 mmol) and water (100 mL). The organic layer was separated, the aqueous layer was extracted with DCM (x 1, 30 mL). The combined organic layers were dried over MgSO₄, filtered and the solvent evaporated under reduced pressure. The residue was purified by flash column chromatography on silica gel using as eluent gradient DCM/MeOH 95/5. The pure fractions were collected and evaporated. The residue was co-evaporated with toluene (x 2) to yield intermediate 51 (220 mg, assumed quant. yield).

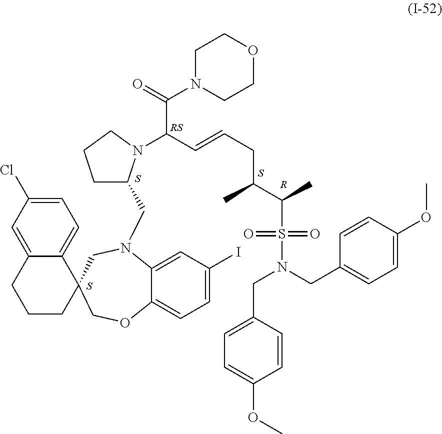

1-propanephosphonic anhydride (514.57 μL, 0.43 mmol) was added dropwise to a stirred solution of I-41 (205 mg, 0.21 mmol), TEA (205.83 μL, 1.48 mmol) and morpholine (82.33 mg, 0.95 mmol) in DCM (6.17 mL) at 0° C. After the addition, the reaction mixture was stirred at room temperature for 2 hours. The reaction mixture was diluted with dichloromethane and poured into water. The aqueous layer was extracted with DCM. The organic layer was dried with MgSO₄ and the solvent evaporated. The residue was purified by flash column chromatography on silica gel using as eluent DCM/MeOH 98/2 to yield intermediate 52 (145 mg, yield 66%).

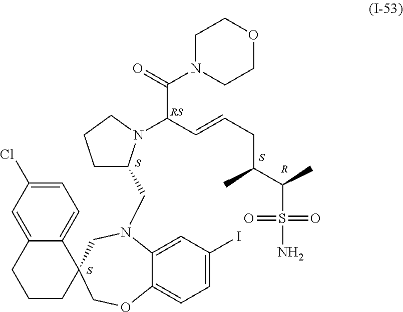

To a solution of I-52 (145 mg, 0.14 mmol) in DCM (1.77 mL) at 0° C. was added dropwise TFA (1.58 mL, 20.68 mmol). After the addition, the reaction mixture was stirred at 10° C. for 16 h. The reaction mixture was diluted with DCM and poured into aqueous NaHCO₃ saturated solution (20 mL). The organic layer was separated, the aqueous layer was extracted with DCM. The combined organic layers were dried over MgSO₄, filtered and the solvent evaporated under reduced pressure. The residue was purified by flash column chromatography on silica gel using as eluent gradient DCM/MeOH 98/2 to yield intermediate 53 (100 mg, 89% yield).

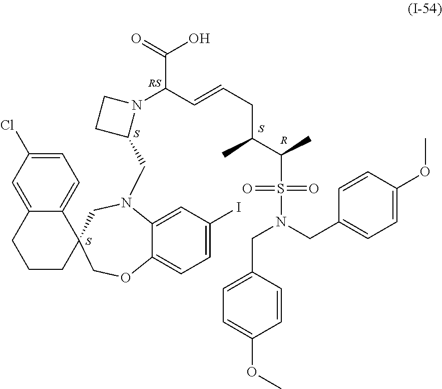

A solution of I-38 (200 mg, 0.37 mmol), I-22 (182.07 mg, 0.37 mmol), glyoxylic acid (54.49 mg, 0.74 mmol) and 3 Å molecular sieves in THE (4.49 mL) was stirred at 50° C. for 5 h. At this stage, MeOH (5 mL) was added and the reaction mixture was stirred at 50° C. for 16 h. The reaction mixture was filtered off and the solvent was evaporated under reduced pressure. The residue was purified together with another batch via preparative HPLC (stationary phase: RP XBridge Prep C18 OBD—10 μm, 50×150 mm, mobile phase: 0.25% NH₄HCO₃ solution in water, MeOH) to yield intermediate 54 (100 mg, 22% yield).

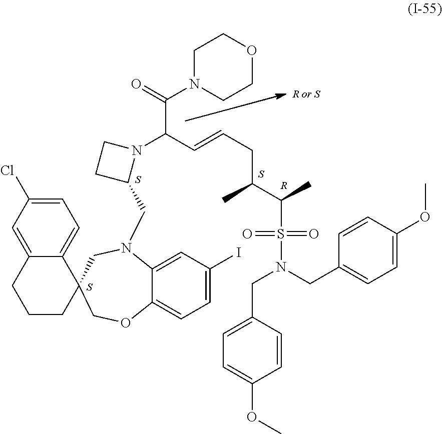

1-propanephosphonic anhydride (254.64 μL, 0.21 mmol) was added dropwise to a stirred solution of I-54 (100 mg, 0.1 mmol), TEA (101.86 μL, 0.73 mmol) and morpholine (40.74 mg, 0.47 mmol) in DCM (3.06 ml) at 0° C. After the addition, the reaction mixture was stirred at room temperature for 2 hours. The reaction mixture was diluted with dichloromethane and poured into water. The aqueous layer was extracted with DCM. The organic layer was dried with MgSO₄ and the solvent evaporated under reduced pressure to give intermediate 55 (120 mg, assumed quant. yield).

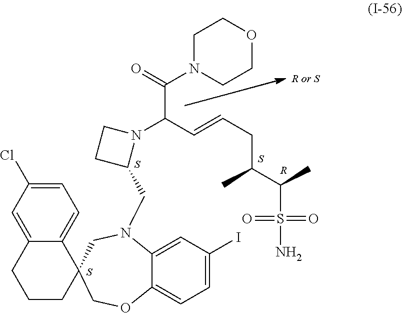

To a solution of I-55 (103.75 mg, 0.1 mmol) in DCM (1.28 mL) at 0° C. was added dropwise TFA (1.15 mL, 15 mmol). After the addition, the reaction mixture was stirred at 10° C. for 16 h. The RM was poured cold into a mixture of DCM (30 mL) and aqueous NaHCO₃ saturated solution (20 mL). The organic layer was separated, the aqueous layer was extracted with DCM (30 mL, ×1). The combined organic layers were dried over MgSO₄, filtered and the solvent evaporated under reduced pressure. The residue was purified via preparative HPLC (stationary phase: RP XBridge Prep C18 OBD—10 μm, 50×150 mm, mobile phase: 0.25% NH₄HCO₃ solution in water, CH₃CN) and the product was co-evaporated with toluene (x 2) to yield intermediate 56 (45 mg, 56% yield).

### Preparation of Compounds

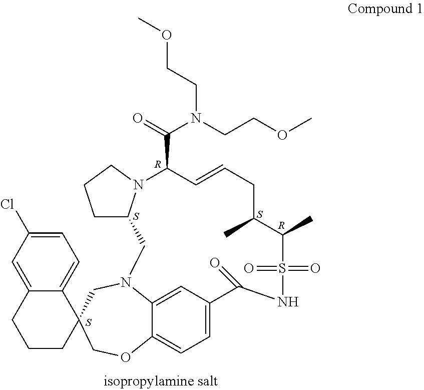

I-43 (90 mg, 0.105 mmol), [1,1′-bis(diphenylphosphino)ferrocene]-dichloropalladium(II) (11.5 mg, 0.0157 mmol), 1,8-diazabicyclo[5.4.0]undec-7-ene (48 mg, 0.315 mmol) and dry THE (43 mL) were stirred at room temperature in a pressure vessel. The vessel was filled with 50 bar of CO gas and stirred at 100° C. for 16 h. The reaction mixture was poured into water and extracted with EtOAc. The organic layer was dried over MgSO₄ and the solvent evaporated under reduced pressure. The resulting residue was purified via preparative HPLC (stationary phase: RP XBridge Prep C18 OBD—10 μm, 50×150 mm, mobile phase: 0.25% NH₄HCO₃ solution in water, CH₃CN). The resulting product was purified via preparative SFC (stationary phase: Chiralcel Diacel IH 20×250 mm, mobile phase: CO₂, EtOH+0.4 iPrNH₂) to yield Compound 1 (26 mg, 30% yield) as a white solid (isopropylamine salt).

¹H NMR (400 MHz, CHLOROFORM-d) δ ppm 1.02 (d, J=6.4 Hz, 3H) 1.22 (d, J=6.5 Hz, 3H) 1.38 (d, J=7.1 Hz, 4H) 1.51-1.77 (m, 3H) 1.80-1.99 (m, 3H) 2.02-2.24 (m, 4H) 2.82 (br dd, J=32.5, 5.1 Hz, 5H) 3.30-3.38 (m, 5H) 3.41 (s, 4H) 3.50-3.63 (m, 5H) 3.65 (s, 1H) 3.97 (br d, J=12.4 Hz, 3H) 4.03-4.19 (m, 4H) 4.61 (d, J=8.0 Hz, 1H) 5.78 (br s, 1H) 5.83 (br d, J=8.0 Hz, 1H) 6.89 (d, J=8.0 Hz, 1H) 7.02 (s, 1H) 7.08 (d, J=2.1 Hz, 1H) 7.14 (d, J=1.0 Hz, 1H) 7.17-7.22 (m, 1H) 7.71 (d, J=8.5 Hz, 1H); Rt=2.15 min, MS (ESI) m/z 757 [M+H], LCMS Method: 1

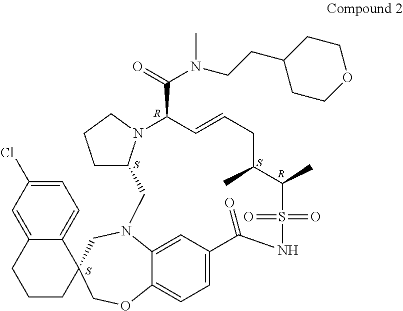

I-45 (195 mg, 0.225 mmol), [1,1′-bis(diphenylphosphino)ferrocene]dichloropalladium(II) (16.45 mg, 0.0225 mmol), 1,8-diazabicyclo[5.4.0]undec-7-ene (102.68 mg, 1.019 g/mL, 0.674 mmol) and dry THF (35 mL,) were stirred at room temperature in a pressure vessel. The vessel was filled with 50 bar of CO gas and stirred at 100° C. for 16 h. The solvent from the reaction mixture was evaporated under reduced pressure. The resulting residue was purified via preparative HPLC (stationary phase: RP XBridge Prep C18 OBD—10 μm, 30×150 mm, mobile phase: 0.5% NH₄OAc solution in water+10% CH₃CN, CH₃CN). The pure fractions were collected, and the solvent evaporated under reduced pressure. The residue was taken up into water, basified with NaHCO₃ and extracted with DCM. The organic layer was dried with MgSO₄, filtered and the solvent evaporated under reduced pressure to yield Compound 2 (55 mg, 32% yield).

¹H NMR (400 MHz, CHLOROFORM-d) δ ppm 1.02 (d, J=6.7 Hz, 3H) 1.24-1.40 (m, 3H) 1.43 (d, J=7.2 Hz, 3H) 1.47-1.83 (m, 9H) 1.86-2.17 (m, 6H) 2.71-2.82 (m, 3H) 2.88 (br d, J=4.5 Hz, 1H) 2.93 (s, 1H) 3.01-3.12 (m, 1H) 3.20-3.41 (m, 7H) 3.44-3.64 (m, 1H) 3.88-4.05 (m, 5H) 4.12-4.39 (m, 3H) 5.60-5.74 (m, 1H) 5.77-5.94 (m, 1H) 6.86-6.97 (m, 2H) 6.99-7.07 (m, 1H) 7.09 (d, J=2.3 Hz, 1H) 7.20 (dd, J=8.5, 2.3 Hz, 1H) 7.65-7.73 (m, 1H); Rt=2.16 min, MS (ESI) m/z 767 [M+H]⁺, LCMS Method: 1

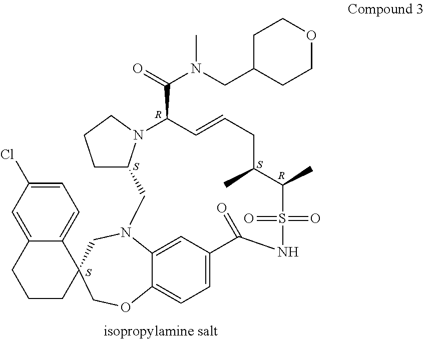

I-47 (90 mg, 0.105 mmol), [1,1′-bis(diphenylphosphino)ferrocene]dichloropalladium(II) (11.6 mg, 0.0158 mmol), 1,8-diazabicyclo[5.4.0]undec-7-ene (48 mg, 0.316 mmol) and dry THF (43 mL) were stirred at room temperature in a pressure vessel. The vessel was filled with 50 bar of CO gas and stirred at 100° C. for 16 h. The reaction mixture was poured into water and extracted with EtOAc. The organic layer was dried over MgSO₄, filtered and the solvent evaporated under reduced pressure. The resulting residue was purified via preparative HPLC (stationary phase: RP XBridge Prep C18 OBD-10 μm, 50×150 mm, mobile phase: 0.25% NH₄HCO₃ solution in water, CH₃CN). The resulting product was purified via preparative SFC (stationary phase: Chiralcel Diacel IH 20×250 mm, mobile phase: CO₂, EtOH+0.4 iPrNH₂) to yield Compound 3 (23 mg, 27% yield) as a white solid (isopropylamine salt).

¹H NMR (400 MHz, CHLOROFORM-d) δ ppm 1.02 (d, J=6.6 Hz, 3H) 1.22-1.46 (m, 7H) 1.46-1.75 (m, 6H) 1.76-1.86 (m, 1H) 1.88-2.00 (m, 3H) 2.01-2.15 (m, 4H) 2.69-2.82 (m, 3H) 2.82-2.99 (m, 2H) 3.02-3.12 (m, 1H) 3.19 (brd, J=7.0 Hz, 1H) 3.27-3.49 (m, 7H) 3.93-4.07 (m, 5H) 4.14-4.25 (m, 2H) 4.30 (d, J=8.6 Hz, 1H) 5.69 (br dd, J=6.9, 4.8 Hz, 1H) 5.79-5.91 (m, 1H) 6.92 (s, 2H) 7.00-7.12 (m, 2H) 7.20 (dd, J=8.5, 2.0 Hz, 1H) 7.66-7.72 (m, 1H); Rt=2.15 min, MS (ESI) m/z 753 [M+H]⁺, LCMS Method: 1

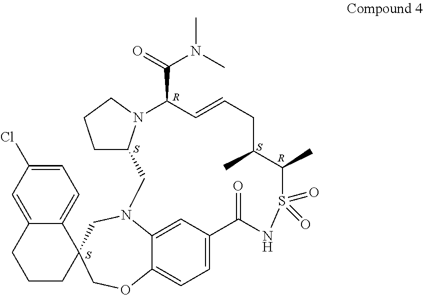

I-49 (180 mg, 0.234 mmol) was dissolved in anhydrous THE (30 mL) in a COware vessel. After degassing with nitrogen, 1,8-diazabicyclo[5.4.0]undec-7-ene [6674-22-2](180 μL, 1.019 g/mL, 1.205 mmol) was added followed by [1,1′-bis(diphenylphosphino)ferrocene]dichloropalladium(II) [72287-26-4](1.8 mg, 0.00246 mmol). The mixture was evacuated and filled with CO (×3) then heated at 80° C. under ca. 30 bar CO pressure during 6 h. Upon cooling to room temperature, the mixture was carefully acidified with 1 M HCl solution until pH reached ca. 5-6. Water (10 mL) and EtOAc (10 mL) were added. The organic layer was separated, washed with brine (10 mL), dried over MgSO₄ and filtered off. Then SiliaMetS DMT (ca. 5 eq) was added and the suspension was stirred 16 h at room temperature, before being filtered over celite and washed with EtOAc. The filtrate was evaporated under reduced pressure to get the crude product as a yellow solid. The crude product was purified via preparative HPLC (Stationary phase: RP XBridge Prep C18 OBD—5 μm, 50×250 mm, mobile phase: 0.5% NH₄OAc solution in water+10% CH₃CN, CH₃CN) to yield Compound 4 (77 mg, yield 49%) as an off-white solid.

¹H NMR (400 MHz, CHLOROFORM-d) δ ppm 1.01 (d, J=6.4 Hz, 3H) 1.42 (br d, J=7.0 Hz, 4H) 1.49-1.66 (m, 2H) 1.71 (dt, J=11.1, 5.7 Hz, 1H) 1.76-1.84 (m, 1H) 1.92 (br s, 2H) 1.99-2.13 (m, 4H) 2.69-2.85 (m, 3H) 2.90 (br dd, J=15.0, 10.3 Hz, 1H) 2.97 (s, 3H) 3.03-3.12 (m, 1H) 3.22-3.39 (m, 5H) 3.91-4.07 (m, 3H) 4.18 (d, J=12.3 Hz, 1H) 4.20-4.27 (m, 1H) 4.37 (d, J=8.4 Hz, 1H) 5.59-5.76 (m, 1H) 5.85 (dd, J=15.6, 8.4 Hz, 1H) 6.94 (s, 1H) 6.92-6.96 (m, 1H) 7.02-7.13 (m, 2H) 7.20 (dd, J=8.5, 2.3 Hz, 1H) 7.69 (d, J=8.6 Hz, 1H); Rt=2.03 min, MS (ESI) m/z 669 [M+H]⁺, LCMS Method: 1

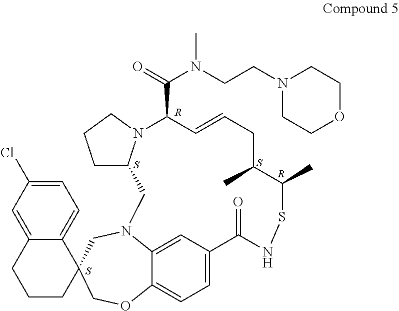

I-51 (220 mg, 0.253 mmol) was dissolved in anhydrous THE (30 mL) in a COware vessel. After degassing with nitrogen, 1,8-diazabicyclo[5.4.0]undec-7-ene [6674-22-2](200 μL, 1.019 g/mL, 1.339 mmol) was added followed by [1,1′-bis(diphenylphosphino)ferrocene]dichloropalladium(II) [72287-26-4](2 mg, 0.00273 mmol). The mixture was evacuated and filled with CO (×3) then heated at 100° C. under ca. 30 bar CO pressure during 6 h. Upon cooling to room temperature, the mixture was carefully acidified with 1 M HCl solution until pH reached ca. 5-6. Water (10 mL) and EtOAc (10 mL) were added. The organic layer was extracted, washed with brine (10 mL), dried over MgSO₄ and filtered off. Then SiliaMet DMT (ca. 5 eq) was added and the suspension was stirred 16 h at room temperature, before being filtered over celite and washed with EtOAc. The filtrate was evaporated under reduced pressure to get the crude product as a yellow solid. The crude product was purified via preparative HPLC (stationary phase: RP XBridge Prep C18 OBD—5 μm, 50×250 mm, mobile phase: 0.5% NH₄OAc solution in water+10% CH₃CN, CH₃CN) to yield the product (98 mg, yield 50%). The product was further purified by flash column chromatography on silica gel using as eluent DCM/MeOH (100:0 to 95:5) to afford Compound 5 (76 mg, yield 39%) as a white solid.

¹H NMR (400 MHz, CHLOROFORM-d) δ ppm 1.03 (d, J=6.6 Hz, 3H) 1.43 (d, J=7.3 Hz, 4H) 1.52-1.75 (m, 3H) 1.76-1.98 (m, 3H) 1.98-2.18 (m, 4H) 2.46-2.63 (m, 5H) 2.65-2.94 (m, 5H) 2.98 (s, 2H) 3.24-3.38 (m, 4H) 3.41-3.77 (m, 6H) 3.82-4.06 (m, 3H) 4.15-4.28 (m, 2H) 4.35 (br d, J=8.1 Hz, 1H) 5.62-5.77 (m, 1H) 5.78-5.95 (m, 1H) 6.88-7.01 (m, 2H) 7.10 (s, 2H) 7.20 (dd, J=8.6, 2.2 Hz, 1H) 7.70 (d, J=8.6 Hz, 1H). (2/1 mixture of rotamers visible on NMR); Rt=2.01 min, MS (ESI) m/z 768 [M+H]⁺, LCMS Method: 1

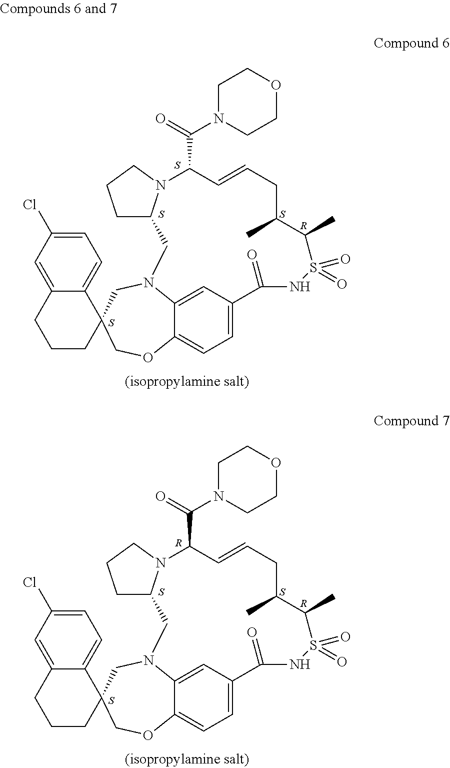

I-53 (100 mg, 0.12 mmol), [1,1′-bis(diphenylphosphino)ferrocene]dichloropalladium(II) (18.04 mg, 0.025 mmol), 1,8-diazabicyclo[5.4.0]undec-7-ene (95.34 mg, 0.63 mmol) and dry THE (25.78 mL) were stirred at room temperature in a pressure vessel. The vessel was filled with 50 bar of CO gas and stirred at 100° C. for 16 h. The reaction mixture was cooled to room temperature and the solvent evaporated under reduced pressure. The residue was purified via preparative HPLC (stationary phase: RP XBridge Prep C18 OBD—10 μm, 50×150 mm, mobile phase: 0.25% NH₄HCO₃ solution in water, MeOH) to yield impure compound 6 and impure compound 7. Compound 6 was purified via preparative SFC (stationary phase: Chiralcel Diacel OJ20×250 mm, mobile phase: CO₂, EtOH+0.4 iPrNH₂) to give pure Compound 6 as (isopropylamine salt (3 mg, yield 3%). Compound 7 was purified via preparative SFC (stationary phase: Chiralcel Diacel OJ 20×250 mm, mobile phase: CO₂, EtOH+0.4 iPrNH₂) to give pure Compound 7 as isopropylamine salt (40 mg, yield 42%).

### Compound 6

¹H NMR (400 MHz, CHLOROFORM-d) δ ppm 0.75-1.01 (m, 2H) 1.13 (br d, J=6.5 Hz, 3H) 1.26 (br s, 3H) 1.37 (br d, J=7.3 Hz, 5H) 1.54-1.65 (m, 2H) 1.67-1.87 (m, 4H) 1.88-2.04 (m, 4H) 2.13 (br s, 1H) 2.53 (br s, 1H) 2.75-2.90 (m, 3H) 2.95 (br s, 1H) 2.99-3.16 (m, 1H) 3.31 (br d, J=14.3 Hz, 2H) 3.35-3.46 (m, 2H) 3.47-3.65 (m, 4H) 3.65-3.86 (m, 4H) 3.88-4.10 (m, 3H) 4.17 (brd, J=11.8 Hz, 1H) 5.55 (brs, 1H) 5.90 (br s, 1H) 6.93 (br d, J=8.2 Hz, 1H) 7.05-7.12 (m, 1H) 7.13-7.24 (m, 2H) 7.69 (br d, J=8.6 Hz, 2H); Rt=2.04 min, MS (ESI) m/z 711 [M+H]⁺, LCMS Method: 1

### Compound 7

¹H NMR (400 MHz, CHLOROFORM-d) δ ppm 1.03 (d, J=6.6 Hz, 3H) 1.21 (d, J=6.4 Hz, 3H) 1.38 (d, J=7.0 Hz, 4H) 1.53-1.76 (m, 3H) 1.77-1.96 (m, 3H) 2.00 (br s, 1H) 2.09-2.29 (m, 3H) 2.78 (br d, J=4.8 Hz, 2H) 2.88 (br d, J=10.1 Hz, 2H) 2.95 (br s, 1H) 3.33 (br d, J=14.1 Hz, 1H) 3.38-3.47 (m, 1H) 3.54 (br d, J=10.1 Hz, 1H) 3.72 (br dd, J=7.6, 5.8 Hz, 6H) 3.83 (br d, J=12.1 Hz, 1H) 3.90-4.04 (m, 4H) 4.17 (d, J=12.3 Hz, 1H) 4.28 (d, J=8.1 Hz, 1H) 5.80 (br s, 1H) 5.86 (br d, J=7.9 Hz, 1H) 6.90 (d, J=8.1 Hz, 1H) 7.04-7.09 (m, 2H) 7.12 (d, J=1.3 Hz, 1H) 7.19 (dd, J=8.5, 2.3 Hz, 1H) 7.70 (d, J=8.4 Hz, 1H); Rt=2.02 min, MS (ESI) m/z 711 [M+H]⁺, LCMS Method: 1

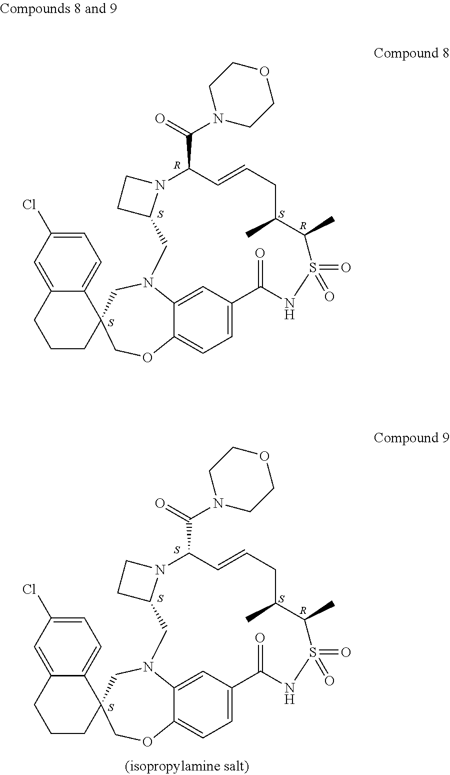

I-56 (45 mg, 0.056 mmol), [1,1′-bis(diphenylphosphino)ferrocene]dichloropalladium(II) (8.26 mg, 0.011 mmol), 1,8-diazabicyclo[5.4.0]undec-7-ene (44.12 mg, 0.29 mmol) and dry THF (11.81 mL) were stirred at room temperature in a pressure vessel. The vessel was filled with 50 bar of CO gas and stirred at 100° C. for 16 h. The solvent from the reaction mixture was evaporated under reduced pressure. The residue was purified via preparative HPLC (stationary phase: RP XBridge Prep C18 OBD—10 μm, 50×150 mm, mobile phase: 0.25% NH₄HCO₃ solution in water, CH₃CN) to yield Compound 8 (23 mg, yield 58%) and impure compound 9. Compound 9 was purified via preparative HPLC (stationary phase: RP XBridge Prep C18 OBD—10 μm, 50×150 mm, mobile phase: 0.25% NH₄HCO₃ solution in water, CH₃CN) then further purified via preparative SFC (stationary phase: Chiralcel Diacel OJ 20×250 mm, mobile phase: CO₂, EtOH+0.4 iPrNH₂) to yield pure Compound 9 as isopropylamine salt (3.3 mg, yield 8%).

### Compound 8

¹H NMR (400 MHz, CHLOROFORM-d) δ ppm 1.03 (d, J=6.4 Hz, 3H) 1.34-1.51 (m, 4H) 1.75-1.88 (m, 1H) 1.90-2.23 (m, 8H) 2.70-2.83 (m, 2H) 3.10-3.31 (m, 3H) 3.44-3.61 (m, 3H) 3.62-3.80 (m, 8H) 3.85 (br d, J=15.2 Hz, 1H) 4.03-4.17 (m, 3H) 4.21 (br d, J=7.2 Hz, 1H) 5.75 (td, J=7.7, 3.7 Hz, 1H) 5.98 (dd, J=15.5, 8.8 Hz, 1H) 6.92-6.96 (m, 1H) 6.99 (br d, J=6.7 Hz, 2H) 7.09 (d, J=2.2 Hz, 1H) 7.17 (dd, J=8.5, 2.2 Hz, 1H) 7.68 (d, J=8.5 Hz, 1H); Rt=1.92 min, MS (ESI) m/z 697 [M+H]⁺, LCMS Method: 2

### Compound 9

¹H NMR (400 MHz, CHLOROFORM-d) δ ppm 0.88 (br s, 1H) 1.10 (br d, J=6.5 Hz, 3 H) 1.20-1.33 (m, 6H) 1.35-1.52 (m, 2H) 1.76-1.89 (m, 2H) 1.92-2.11 (m, 5H) 2.19 (br s, 1H) 2.77 (br s, 3H) 3.03 (br dd, J=15.0, 8.9 Hz, 1H) 3.11-3.26 (m, 3H) 3.37-3.52 (m, 2H) 3.54-3.80 (m, 8H) 3.86 (br d, J=15.0 Hz, 1H) 3.99 (brd, J=7.5 Hz, 1H) 4.06 (s, 1H) 4.21-4.24 (m, 1H) 5.70 (br d, J=15.9 Hz, 1H) 6.08-6.29 (m, 1H) 6.89 (br d, J=8.3 Hz, 1H) 7.08 (d, J=1.8 Hz, 1H) 7.17 (dd, J=8.4, 1.7 Hz, 1H) 7.27-7.32 (m, 1H) 7.47 (br s, 1H) 7.70 (d, J=8.5 Hz, 1H); Rt=2.01 min, MS (ESI) m z 697 [M+H]+, LCMS Method: 1

### Analytical Methods

### LCMS

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc., . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method Codes (Flow expressed in mL/min;
column temperature (T) in ° C.; Run time in minutes)

| Method | | | | | Flow | Run |
|---|---|---|---|---|---|---|
| Code | Instrument | column | mobile phase | gradient | Col T | time |
| 1 | Waters: | Waters: | A: 10 mM | From | 0.6 | 3.5 |
| | Acquity ® | BEH | CH₃COONH₄ | 100% A to | 55 | |
| | UPLC ®- | (1.8 μm, | in 95% H₂O + | 5% A in | | |
| | DAD and | 2.1 * | 5% CH₃CN | 2.10 min, | | |
| | SQD | 100 mm) | B: CH₃CN | to 0% A | | |
| | | | | in 0.90 | | |
| | | | | min, to | | |
| | | | | 5% A | | |
| | | | | in 0.5 min | | |
| 2 | Waters: | Waters: | A: 0.1% | From | 0.6 | 4.5 |
| | Acquity ® | BEH | NH₄HCO₃ | 100% A to | 55 | |
| | UPLC ®- | (1.8 μm, | in 95% H₂O + | 5% A in | | |
| | DAD and | 2.1 * | 5% CH₃CN | 2.10 min, | | |
| | SQD | 100 mm) | B: CH₃CN | to 0% A | | |
| | | | | in 0.9 min, | | |
| | | | | to 5% A | | |
| | | | | in 0.5 min | | |

LCMS results (RT means retention time)

| Compound | |
|---|---|
| number | LCMS results |
| 1 | confirms the MW (Rt: 2.15 min, [M + H]+ 757, LCMS Method 1) |
| 2 | confirms the MW (Rt: 2.16 min, [M + H]+ 767, LCMS Method 1) |
| 3 | confirms the MW (Rt: 2.15 min, [M + H]+ 753, LCMS Method 1) |
| 4 | confirms the MW (Rt: 2.03 min, [M + H]+ 669, LCMS Method 1) |
| 5 | confirms the MW (Rt: 2.01 min, [M + H]+ 768, LCMS Method 1) |
| 6 | confirms the MW (Rt: 2.04 min, [M + H]+ 711, LCMS Method 1) |
| 7 | confirms the MW (Rt: 2.02 min, [M + H]+ 711, LCMS Method 1) |
| 8 | confirms the MW (Rt: 1.92 min, [M + H]+ 697, LCMS Method 2) |
| 9 | confirms the MW (Rt: 2.01 min, [M + H]+ 697, LCMS Method 1) |

### NMR

¹H NMR spectra were recorded on Bruker Avance III and Avance NEO spectrometers. CDCl₃ was used as solvent, unless otherwise mentioned. The chemical shifts are expressed in ppm relative to tetramethylsilane.

### Pharmacological Analysis

### Biological Example 1

Terbium labeled Myeloid Cell Leukemia 1(Mcl-1) homogeneous time-resolved fluorescence (HTRF) binding assay utilizing the BIM BH3 peptide (H₂N—(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

### Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1× assay buffer, where the following ingredients were added fresh to stock buffer: 2 mM dithiothreitol (DTT), 0.0025% Tween-20, 0.1 mg/mL bovine serum albumin (BSA). The 1×Tb-Mcl-1+Cy5 Bim peptide solution was prepared by diluting the protein stock solution using the 1× assay buffer (b) to 25 pM Tb-Mel-1 and 8 nM Cy5 Bim peptide.

Using the Acoustic ECHO, 100 nL of 100× test compound(s) were dispensed into individual wells of a white 384-well Perkin Elmer Proxiplate, for a final compound concentration of 1× and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1×Tb-Mcl-1+Cy5 Bim peptide solution. The plate was centrifuged with a cover plate at 1000 rpm for 1 minute, then incubated for 60 minutes at room temperature with plates covered.

The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 μs).

### Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

% inhibition(((NC−IC)−(compound−IC))(NC−IC))*100

where IC (inhibitor control, low signal)=mean signal of 1×Tb-MCl-1+Cy5 Bim peptide+inhibitor control or 100% inhibition of Mcl-1; NC (neutral control, high signal)=mean signal 1×Tb-MCl-1+Cy5 Bim peptide with DMSO only or 0% inhibition An 11-point dose response curve was generated to determine IC₅₀ values (using GenData) based on the following equation:

$$Y = Bottom + ( Top - Bottom ) / ( 1 + 10 \hat{} ( ( log {IC}_{50} - X ) * HillSlope ) )$$

where Y=% inhibition in the presence of X inhibitor concentration; Top=100% inhibition derived from the IC (mean signal of Mcl-1+inhibitor control); Bottom=0% inhibition derived from the NC (mean signal of Mcl-1+DMSO); Hillslope=Hill coefficient; and IC₅₀=concentration of compound with 50% inhibition in relation to top/neutral control (NC).

$${K}_{i} = {IC}_{50} / ( 1 + [ L ] / {K}_{d} )$$

$$In this assay [ L ] = 8 nM and {K}_{d} = 10 nM$$

Representative compounds of the present invention were tested according to the procedure as described above, with results as listed in the Table below (n.d. means not determined).

| Compound | Tb-MCL1 Kᵢ (nM) |
|---|---|
| 1 | 0.030 |
| 2 | 0.058 |
| 3 | 0.027 |
| 4 | 0.142 |
| 5 | 0.024 |
| 6 | nd |
| 7 | 0.089 |
| 8 | 0.705 |
| 9 | 0.196 |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and −7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal.

This assay uses the MOLP-8 human multiple myeloma cell line, which is sensitive to MCL-1 inhibition.

### Materials:

Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| Product | Units | Storage |
|---|---|---|
| RPMI1640 (no L-Glutamine, no | 500 mL | 4° C. |
| phenol red) | | |
| Foetal Bovine Serum (FBS) (Heat | 500 mL | 4° C. |
| inactivated) | | |
| L-Glutamine (200 mM) | 100 mL | −20° C. |
| Gentamicin (50 mg/mL) | 100 mL | 4° C. |
| Caspase 3/7 Detection kit | 100 mL | −20° C. |
| | 10 × 10 mL | |

### Cell Culture Media:

| MOLP8 | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |
| Assay media | | |
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

### Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

### Caspase-Glo Reagent:

The assay reagent was prepared by transferring the buffer solution to the substrate vial and mixing. The solution may be stored for up to 1 week at 4° C. with negligible loss of signal.

### Assay Procedure:

Compounds were delivered in assay-ready plates (Proxiplate) and stored at −20° C.

Assays always include 1 reference compound plate containing reference compounds. The plates were spotted with 40 nL of compounds (0.5% DMSO final in cells; serial dilution; 30 μM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 μM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:
LC=median of the Low Control values
   =Central Reference in Screener
   =DMSO
   =00%
HC=Median of the High Control values
   =Scale Reference in Screener
   =30 μM of positive control
   =100% apoptosis induction

% Effect (AC₅₀)=100−((sample−LC)/(HC−LC))*100

% Control=(sample/HC)*100

% Control min=((sample−LC)/(HC−LC))*100

TABLE
Measured AC₅₀ for Representative Compounds of Formula
(I). Averaged values are reported over all runs
on all batches of a particular compound.

| Compound | MOLP8 AC₅₀ (μM) |
|---|---|
| 1 | 0.056 |
| 2 | 0.13 |
| 3 | 0.081 |
| 4 | 0.18 |
| 5 | 0.18 |
| 6 | 1.95 |
| 7 | 0.36 |
| 8 | 0.34 |
| 9 | 0.66 |

1. A compound of Formula (I) 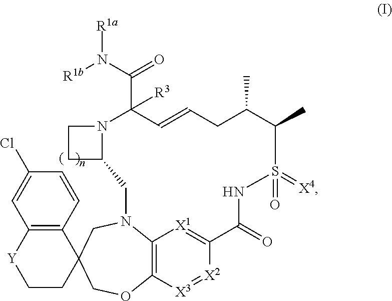
wherein
R^{1a} and R^{1b} are each independently selected from the group consisting of hydrogen, C₁₋₆alkyl, C₂₋₆alkenyl, C₂₋₆alkynyl, C₃₋₇cycloalkyl, C₃₋₇cycloalkenyl, Het¹, Ar¹, Het², and Cy¹, wherein said C₁₋₆alkyl, C₂₋₆alkenyl, C₂₋₆alkynyl, C₃₋₇cycloalkyl or C₃₋₇cycloalkenyl is optionally substituted with one or two R²,
or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{c}, CN, halo, CF₃, and C₁₋₄alkyl optionally substituted with one substituent selected from the group consisting of OR^{f}, SR^{f}, CN and halo;
or R^{1a} and R^{1b} are taken together to form together with the N-atom to which they are attached a 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of oxo, OR^{f}, SR^{f}, NR^{d}R^{e}, CN, halo, CF₃, and C₁₋₄alkyl optionally substituted with one substituent selected from the group consisting of OR^{f}, SR^{f}, CN and halo;
each R² is independently selected from the group consisting of OR^{f}, SR^{f}, CN, halo, CF₃, NR^{m}Rⁿ, SO₂R^{c}, C(═O)R^{c}, C(═O)OR^{d}, C(═O)NR^{d}R^{c}, SO₂NR^{d}R^{c}, C₃₋₇cycloalkyl, C₃₋₇cycloalkenyl, Het¹, Ar¹, Het², and Cy¹ wherein said C₃₋₇cycloalkyl or C₃₋₇cycloalkenyl is optionally substituted with one or two substituents each independently selected from the group consisting of OR^{f}, SR^{f}, CN, halo and NR^{d}R^{c};
R^{c} is selected from the group consisting of C₁₋₆alkyl, C₃₋₇cycloalkyl, Het¹, Ar¹ and Het²,
R^{m} and Rⁿ are each independently selected from the group consisting of hydrogen, methyl, C₂₋₇alkyl, C₃₋₇cycloalkyl, Het¹, Ar¹, and let², wherein said C₂₋₇alkyl or C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;
R^{d} and R^{c} are each independently selected from the group consisting of hydrogen, methyl, C₂₋₇alkyl, C₃₋₇cycloalkyl, Het¹, Ar¹, and Het², wherein said C₂₋₇alkyl or
C₃₋₇cycloalkyl is optionally substituted with one or two substituents each independently selected from the group consisting of ORⁱ, SRⁱ, NR^{g}R^{h}, CN, halo, and C₁₋₄alkyl optionally substituted with one substituent selected from the group consisting of ORⁱ, SRⁱ, CN, NR^{g}R^{h} and halo;
or R^{d} and R^{e} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
or R^{d} and R^{e} are taken together to form together with the N-atom to which they are attached a fused 6- to 11-membered bicyclic fully saturated heterocyclyl containing one N-atom and optionally one or two additional heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
n is 1 or 2;
R^{f} is selected from the group consisting of hydrogen, C₁₋₆alkyl, CF₃, C₃₋₇cycloalkyl, Het¹, Ar¹, Het², wherein said C₁₋₆alkyl or C₃₋₇cycloalkyl is optionally substituted with one substituent selected from the group consisting of ORⁱ, SRⁱ, CN, halo, NR^{m}Rⁿ, SO₂R^{c}, C(═O)R^{c}, C(═O)OR^{d}, C(═O)NR^{d}R^{c}, SO₂NR^{d}R^{e}, C₃₋₇cycloalkyl, Het¹, Ar¹ and Het²;
R^{g} and R^{h} are each independently selected from the group consisting of hydrogen, C₁₋₆alkyl and C₃₋₇cycloalkyl;
or R^{g} and R^{h} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;
Het¹ represents a 4- to 7-membered monocyclic fully saturated heterocyclyl containing one or two heteroatoms each independently selected from O, S, and N, wherein said S-atom might be substituted to form S(═O) or S(═O)₂, and wherein said heterocyclyl is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Het² represents a 5- to 6-membered monocyclic aromatic ring containing one, two, three or four heteroatoms each independently selected from O, S, and N, wherein said S-atom might be substituted to form S(═O) or S(═O)₂, and wherein said aromatic ring is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Cy¹ represents a 6- to 11-membered bicyclic fully saturated ring system optionally containing one or two heteroatoms each independently selected from O, S, and N, wherein said S-atom might be substituted to form S(═O) or S(═O)₂, and wherein said ring system is optionally substituted with one or two substituents each independently selected from the group consisting of ORⁱ, SRⁱ, NR^{j}R^{k}, CN, halo, CF₃, and C₁₋₄alkyl optionally substituted with one substituent selected from the group consisting of ORⁱ, SRⁱ, CN and halo;
Ar¹ represents phenyl optionally substituted with one or two substituents each independently selected from the group consisting of ORⁱ, SRⁱ, NR^{g}R^{h}, CN, halo, CF₃, and C₁₋₄alkyl optionally substituted with one substituent selected from the group consisting of ORⁱ, SRⁱ, CN and halo;
Rⁱ represents hydrogen, C₁₋₆alkyl or C₃₋₇cycloalkyl;
R^{j} and R^{k} are each independently selected from the group consisting of hydrogen, C₁₋₆alkyl and C₃₋₇cycloalkyl;
R³ represents hydrogen, C₁₋₄alkyl or C₁₋₄alkyl-OH;
R⁴ represents hydrogen or methyl;
R⁵ represents —(C═O)-phenyl, —(C═O)-Het⁴ or —(C═O)-Het³; wherein said phenyl, Het³ or Het⁴ are optionally substituted with one or two substituents selected from methyl or methoxy;
Het⁴ represents a C-linked 4- to 7-membered monocyclic fully saturated heterocyclyl containing one or two heteroatoms each independently selected from O, S, and N; wherein said S-atom might be substituted to form S(═O) or S(═O)₂;
Het³ represents a C-linked 5- or 6-membered monocyclic aromatic ring containing one, two or three heteroatoms each independently selected from O, S, and N;
Y represents O or CH₂;
X¹ represents CR⁶;
X² represents CR⁷;
X³ represents CR⁸;
R⁶, R⁷ and R⁸ each independently represent hydrogen, fluoro or chloro;
X⁴ represents O or NR⁵;
or a pharmaceutically acceptable salt, or a solvate thereof.

2. The compound according to claim 1, wherein
R^{1a} and R^{1b} represent C₁₋₆alkyl optionally substituted with one or two R²;
or R^{1a} and R^{1b} are taken together to form together with the N-atom to which they are attached a 4- to 7-membered monocyclic fully saturated heterocyclyl containing one N-atom and optionally one additional heteroatom selected from O, S, and N, wherein said S-atom might be substituted to form S(═O) or S(═O)₂;
each R² is independently selected from the group consisting of OR^{f} and Het¹;
n is 1 or 2;
R^{f} represents C₁₋₆alkyl;
Het¹ represents a 4- to 7-membered monocyclic fully saturated heterocyclyl containing one or two heteroatoms each independently selected from O, S, and N, wherein said S-atom might be substituted to form S(═O) or S(═O)₂;
R³ represents hydrogen;
R⁴ represents methyl;
Y represents CH₂;
X¹ represents CR⁶;
X² represents CR⁷;
X³ represents CR⁸;
R⁶, R⁷ and R⁸ represent hydrogen;
X⁴ represents O.

3. The compound according to claim 1, wherein R³ represent hydrogen.

4. The compound according to claim 1, wherein R⁴ represent methyl.

5. The compound according to claim 1, wherein R⁴ represent methyl; and X⁴ represents O.

6. The compound according to claim 1, wherein Y represents CH₂.

7. The compound according to claim 1, wherein n represents 2.

8. The compound according to claim 1, wherein n represents 1.

9. A pharmaceutical composition comprising a compound as claimed in claim 1 and a pharmaceutically acceptable carrier or diluent.

10. A process for preparing a pharmaceutical composition comprising mixing a pharmaceutically acceptable carrier with a therapeutically effective amount of a compound according to claim 1.

11. (canceled)

12. (canceled)

13. (canceled)

14. A method of treating cancer, comprising administering to a subject in need thereof, a therapeutically effective amount of a compound as claimed in claim 1 or a pharmaceutical composition comprising the compound.

15. The method according to claim 14, wherein cancer is selected from prostate, lung, pancreatic, breast, ovarian, cervical, melanoma, B-cell chronic lymphocytic leukemia (CLL), acute myeloid leukemia (AML), and acute lymphoblastic leukemia (ALL).
